(19) United States
(12) Patent Application Publication
(10) Pub. No.: US 2008/0153078 A1
(43) Pub. Date: Jun. 26, 2008

(54) SYSTEM FOR ISOLATING BIOMOLECULES FROM A SAMPLE

(76) Inventors: Jeffrey C. BRAMAN, Carlsbad, CA (US); Lee Scott Basehore, Lakeside, CA (US); Natalia Novoradovskaya, San Diego, CA (US); James Chang, Carlsbad, CA (US)

Correspondence Address: LATIMER, MAYBERRY & MATTHEWS IP LAW, LLP, Suite 106, 13873 Park Center Road, Herndon, VA, 20171, US

(21) Appl. No.: 11/959,587

(22) Filed: Dec. 19, 2007

Related U.S. Application Data
(63) Continuation-in-part of application No. 11/764,117, filed on Jun. 15, 2007.
(60) Provisional application No. 60/814,622, filed on Jun. 15, 2006.

(51) Int. Cl. A01N 1/02; C12M 1/00

(52) U.S. Cl. 435/2; 435/306.1

(57) ABSTRACT

The present invention provides an automated system for purification of a substance of interest. The system generally comprises an instrument for moving fluids through the system, a reagent pack for storing fluids, and a purification cartridge. The cartridge comprises two filtration units for binding substances based on different physical properties. The cartridge also comprises rotary valves for control of movement of fluids on the cartridge. In preferred embodiments, the system is useful for purifying RNA from blood samples.

### CROSS-REFERENCE TO RELATED APPLICATIONS

This application is a continuation-in-part of U.S. patent application Ser. No. 11/764,117, filed 15 Jun. 2007, which claims the benefit of U.S. provisional patent application No. 60/814,622, filed 15 Jun. 2006. The entire disclosures of these prior applications are incorporated herein by reference.

### BACKGROUND OF THE INVENTION

1. Field of the Invention

The present invention relates to the fields of biology, sample analysis, and health care. More specifically, the invention relates to isolation and purification of biological molecules from samples. While applicable to an unlimited number of sample types, the invention is particularly well suited for isolating and purifying nucleic acids, proteins, and other biomolecules from cells found in blood and blood products.

2. Description of Related Art

Isolation of biological molecules, such as DNA, RNA, proteins, and other cellular components, and their subsequent analysis, is a fundamental part of molecular biology and biochemistry. For example, analysis of nucleic acids is used to identify organisms or specific cells in a sample, and used in gene expression studies in both basic research and in the medical field of diagnostics. For example, gene expression studies are used to identify genes involved in certain diseases and disorders, and are used to determine the effect of certain substances (e.g., drugs) on expression of genes. The yield and quality of the nucleic acids isolated and purified from a sample has a critical effect on the success of any subsequent analyses.

Isolation of biological molecules from a cell found in a sample usually involves lysing the cells in the biological sample by, for example, mechanical action and/or chemical action, followed by purification of the molecules of interest, such as nucleic acids or proteins. Purification of nucleic acids has traditionally been performed using cesium chloride density gradient centrifugation or extraction with phenol-chloroform. In a typical final step in these methods, ethanol precipitation is used to concentrate the nucleic acids, which results in isolation of the target nucleic acid, but often with low yields of the isolated nucleic acids. These traditional methods are time-consuming, complicated, and, in some cases, hazardous.

The traditional methods used to isolate nucleic acids have been largely supplanted by methods that involve preferential binding of nucleic acids to solid supports, followed by release of the nucleic acid after washing away contaminating material. For example, U.S. Pat. No. 5,234,809 to Boom et al. describes the principle of adsorption of nucleic acids to silica matrices in the presence of chaotropic salts. The method of nucleic acid purification disclosed in this patent eliminates organic solvent extractions and ethanol precipitations previously performed in the art for nucleic acid purifications. Biological molecules purified or isolated using this method, such as nucleic acids isolated by the method, can have high yields and can be of high quality. Another advantage of using a chaotropic salt in the mixture is that the salt inhibits the action of ribonucleases (RNases).

Use of solid supports for binding nucleic acids is well documented in the art. Numerous solid support materials have been shown to be suitable for binding of DNA and RNA. For example, the usefulness of glass for binding of nucleic acids has been known for some time. In work reported in 1979, Vogelstein and Gillespie disclosed the use of glass beads and chaotropic salts for binding of nucleic acids (B. Vogelstein and D. Gillespie, PNAS 76:615-619, 1979).

Some nucleic purification methods take advantage of the discovery that single-stranded and double-stranded nucleic acids can differentially bind to a mineral substrate in the presence of an organic solvent and chaotropic salts. This characteristic of nucleic acids was first noted with ethanol (see, for example, U.S. Pat. No. 6,180,778) and subsequently with other organic solvents (see, for example, U.S. patent application Ser. Nos. 11/688,652 and 11/688,662, incorporated herein by reference). More specifically, it has been found that single-stranded nucleic acid molecules can bind to a mineral substrate in the presence of chaotropic salts and organic solvent at certain concentrations. As an example, detergent-lysed cells (e.g., mammalian cells, such as those from whole blood or plasma and those cultured in flasks) can be mixed with chaotropic salt and glass fiber filters to capture genomic DNA on the glass fiber filter, while allowing RNA to pass through. Addition of appropriate amounts of organic solvent to the flow-through mixture allows RNA to bind to glass substrates, such as glass fiber. Among other things, this discovery can be used to preferentially separate single-stranded nucleic acids from double-stranded nucleic acids.

Microporous filter-based techniques have surfaced as tools for the purification of genomic DNA as well as a whole multitude of nucleic acids. The advantage of filter-based matrices are that they can be fashioned into many formats that include tubes, spin tubes, sheets, and microwell plates. Microporous filter membranes as purification support matrices have other advantages within the art. For example, they provide a compact, easy to manipulate system allowing for the capture of the desired molecule and the removal of unwanted components in a fluid phase at higher throughput and faster processing times than possible with column chromatography. This feature is due at least in part to the fast diffusion rates possible on filter membranes. Nucleic acid molecules have been captured on filter membranes, generally either through simple adsorption or through a chemical reaction between complementary reactive groups present on the filter membrane or on a filter bound ligand resulting in strong interaction between the ligand and the desired nucleic acid.

Porous filter membrane materials used for non-covalent nucleic acid immobilization include materials such as nylon, nitrocellulose, hydrophobic polyvinylidinefluoride (PVDF), and glass microfiber. A number of methods and reagents have also been developed to allow the direct coupling of nucleic acids onto solid supports, such as oligonucleotides and primers (e.g., J. M. Coull et al., Tetrahedron Lett. 27:3991; B. A. Conolly, Nucleic Acids Res. 15:3131, 1987; B. A. Conolly and P. Rider, Nucleic Acids Res. 12:4485, 1985; and Yang et al., PNAS 95:5462-5467). The use of ultraviolet (UV) radiation to cross-link nucleic acids to nylon membranes has also been reported (Church et al., PNAS 81:1991, 1984; Khandjian et al., Anal. Biochem 159:227, 1986).

More recently, glass microfiber, has been shown to specifically bind nucleic acids from a variety of nucleic acid containing sources very effectively (See, e.g., M. Itoh et al., Nucl. Acids Res. 25:1315-1316, 1997; and B. Andersson et al., BioTechniques 1022:1022-1027, 1996). According to these researchers, using a variety of solution components, nucleic acids will bind to glass or silica with high specificity.

In addition, U.S. Pat. Nos. 5,652,141 and 6,020,186 teach a method of isolating nucleic acids from cells by immobilizing the cells in a porous matrix, lysing the cells under conditions where the nucleic acids are retained on the matrix surface, and eluting the nucleic acids. In addition, U.S. Pat. Nos. 5,187,083 and 5,234,824 describe a method for rapidly obtaining substantially pure DNA from a biological sample containing cells. According to the disclosed method, the membranes of the cells are gently lysed to yield a lysate containing genomic DNA in a high molecular weight form. The lysate is applied to a porous filter under conditions wherein the lysate is removed and the DNA is trapped. The DNA is released from the filter using an aqueous solution. Further, U.S. Pat. No. 6,958,392 teaches a method of isolating nucleic acid from a cell sample wherein cells are applied to a filter and are retained. The cells are lysed on the filter to form a cell lysate containing nucleic acid. The cell lysate is removed from the filter and the DNA is retained. Subsequently, the DNA is eluted from the filter. This patent further teaches a device useful for extraction of a sample, for example blood, wherein the device consists of a body, an inlet, and an outlet, disposed between which is a filter. The filter is preferably disposed between a filter support or frit and a filter retaining member for retaining the filter in place.

U.S. Pat. Nos. 5,496,562, 5,756,126, and 5,807,527 demonstrate that nucleic acids or genetic material can be immobilized to a cellulosic-based dry solid support or filter (FTA filter). The solid support described is conditioned with a chemical composition that is capable of carrying out several functions: (i) lyse intact cellular material upon contact, thus releasing genetic material, (ii) enable and allow for conditions that facilitate genetic material immobilization to the solid support, (iii) maintain the immobilized genetic material in a stable state without damage due to mechanical shear, endonuclease activity, UV interference, and microbial attack, and (iv) maintain the genetic material as a support-bound molecule that is not removed from the solid support during any down stream processing (as demonstrated by Del Rio et al., BioTechniques 20:970-974, 1995). However, this reference recognizes that nucleic acid or genetic material applied to, and immobilized to, FTA filters cannot be simply removed or eluted from the solid support once bound. This shortcoming is a major disadvantage for applications where several downstream processes are required from the same sample.

Membranes for binding nucleic acids have been incorporated into cartridges or other multi-part units. For example, U.S. patent application publication number 2005/0112656 discloses a cartridge for isolation and purification of nucleic acids comprising a nucleic acid adsorbing porous membrane in a container having at least two openings. The nucleic acid adsorbing porous membrane is characterized by adsorbing nucleic acid through non-ionic associations. This patent application also teaches that a porous membrane preferably has a hydrophilic group and is formed by treating or coating the membrane.

Further, U.S. patent application publication number 2006/0051799 describes a cartridge for separating and purifying nucleic acids, where the cartridge comprises a solid phase, a container with at least two openings for placing the solid phase in, and a pressure difference-generating apparatus connected to one of the openings of the container. The cartridge is used for separating and purifying nucleic acid according to a method that requires a step of vortexing, mixing with inversion, or pipetting.

In addition, U.S. patent application publication number 2006/006491 teaches a microdevice for performing a method of separating and purifying of a nucleic acid. The device comprises at least one opening, and at least one microchannel with a diameter of 1 mm or less for passing a sample solution through.

RNA is an important diagnostic tool in gene expression or regulation studies. For example, it can be used in expression profiling or DNA microarrays as an indicator of cell response to certain environmental changes, such as addition of a particular pharmaceutical compound, RNA can also be used for cDNA generation, reverse transcription PCR (RT-PCR), and Northern blot analysis, among other methods. The quality of nucleic acids, such as RNA or DNA, obtained from a nucleic acid isolation method is important in the success of most subsequent molecular biology analyses. The quality of RNA obtained from a particular method depends in part on the ability of that method to inactivate or remove RNases. Unlike DNA molecules, which are relatively stable, RNA molecules are more susceptible to degradation due to the ability of the 2′ hydroxyl groups adjacent to the phosphodiester linkages in RNA to act as intramolecular nucleophiles in both base- and enzyme-catalyzed hydrolysis. Whereas deoxyribonucleases (DNases) require metal ions for activity and therefore can be inactivated by chelating agents, many RNases bypass the need for metal ions by taking advantage of the 2′ hydroxyl group as a reactive species. Indeed, bacterial mRNAs have an extremely short half-life in vivo, such as on the order of only a few minutes. Generally, eukaryotic mRNAs have a longer half-life and are stable for several hours in vivo. However, when cell lysis occurs, eukaryotic mRNAs are no longer in a protected environment and can have a very short lifespan. The ability of a method to reduce the amount of time that RNases are in contact with the RNA molecules affects the quality of RNA purified from a method. An automated RNA purification method is generally faster than a manual method and therefore, less likely to cause RNA degradation. A fully automated method that starts from a sample of whole blood or blood plasma and results in a finished product of isolated RNA without human intervention also has the advantage of not coming into contact with RNases from human fingers or dust in the environment during the purification process. Additionally, an automated method to isolate nucleic acids likely is more reproducible than non-automated procedures that depend on the handling skills of a particular user and the delays that may occur between multiple steps when a user is carrying out several procedures in the laboratory at one time.

Components of blood include blood plasma, platelets, white blood cells, and red blood cells. Plasma is the protein-containing fluid portion of the blood in which the blood cells and platelets are normally suspended. Serum is the fluid that remains after blood is allowed to clot and the clot is removed. Serum and plasma differ only in their content of fibrinogen and other minor components, which are mostly removed in the clotting process. Platelets are minute, irregularly shaped disklike cytoplasmic bodies found in blood plasma that promote blood clotting. Cells of mammalian blood include nucleated leukocytes (white blood cells), nucleated immature red blood cells (reticulocytes), and non-nucleated mature erythrocytes (red blood cells). Leukocytes constitute an important part of the defense and repair mechanism in the body. In general, there are two varieties of leukocytes, termed granular and agranular. Granular leukocytes (granulocytes) include phagocytic cells that engulf debris and bacteria. Agranular cells include lymphocytes, which are of two major classes, B cells and T cells, and play a major role in the immune system. Erythrocytes contain hemoglobin, the protein that carries oxygen and carbon dioxide in the blood.

Blood contains large quantities of erythrocytes compared to leukocytes. Generally, it is difficult to isolate RNA from whole blood because of the presence of large amounts of RNases from granulocytes and red blood cells. Assay procedures are usually labor-intensive and involve careful handling that is essential to eliminate RNAse activity. Purification of nucleic acids from a complicated mixture such as whole blood has been disclosed, such as in U.S. Pat. No. 6,958,392 (see above), which provides a method to purify DNA from whole blood, and in U.S. patent application publication number 2006/0199212, in which mRNA is purified from whole blood using oligo-(dT). However, these disclosures do not contain an automated system for isolation of nucleic acids and are thus time-consuming and rely on a relatively high level of expertise by the practitioner.

One method of obtaining nucleic acids from blood cells includes using filters to selectively remove leukocytes from blood. Commercially available leukodepletion filters are often made of glass fibers, polyester 20 fibers, or a combination of the two types of fibers. One such commercially available leukodepletion filter, the r\LS leukodepletion filter media (HemaSure, Inc.), for example, combines a matrix of fibers, such as glass fibers, with components, such as a highly fibrillated fibers or particles comprising a polyacrylonitrile copolymer having a specific surface area greater than 100 m²/g and an average diameter of less than 0.05 micrometers (um), and, optionally, a binder, such as a polyvinyl alcohol or its derivative. This filter is capable of removing at least 99.99% of the leukocytes from a unit of blood product to provide a leukodepleted blood product. Other commercial leukodepletion filters are available from manufacturers, such as the Pall Purecell LRF High Efficiency Leukocyte Reduction Filtration System (Pall Corporation) and leukoreduction products by Baxter Healthcare Corporation (Fenwal Division)/Asahi Medical Corporation.

It is known in the art that blood samples can be processed using filter paper and a chelating resin, such as Chelex-100. In general, these methods include applying blood to a filter paper disc, adding the chelating resin, and incubating the mixture at high temperatures to elute the DNA that is bound to the filter paper.

In addition, Baker et al. describes a method of purifying DNA from blood samples. According to this method, blood is mixed with a hypotonic solution and is filtered by using a vacuum under conditions wherein the white blood cells are captured within a glass fiber filter matrix. The white blood cells are lysed and the DNA is released from the cells becoming trapped around and/or within the fibers of the filter matrix. DNA is eluted by incubation at high temperatures followed by a vacuum process (Baker et al., BioTechniques 31:142-145, 2001).

Attempts have been made to automate parts of nucleic acid purification techniques, such as dispensing reagents, diluting solutions, and aspiration and mixing of liquids. For example, U.S. Pat. No. 5,104,621 discloses a robotic system that has interchangeable tools for permitting automated procedures in place of manual procedures, according to a computer program that is entered by the user. Such inventions, however, have been designed to be flexible and do not specifically provide for rapid isolation of nucleic acids from whole blood. As such, these robotic systems cannot be considered to be fully automated for purification of molecules from blood or blood products because they may require pretreatment before adding blood samples to the machine or require other steps to be performed during nucleic acid isolation. In addition, the user has to determine the computer program that will be used for nucleic acid isolation as well as the hardware to achieve the purification.

U.S. application publication number 2003/0027203 purports to disclose a fully automated system in which whole blood is mixed with lysis solution, and the mixture is then passed through a filter capable of binding nucleic acids. In subsequent steps, the filter is washed and the nucleic acids are eluted. However, when purifying nucleic acids from blood samples, it is important to remove red blood cells from the solution to avoid heme contamination from hemoglobin, which can inhibit subsequent analyses (see, for example, Akane, A., K. Matsubara, H. Nakamura, S. Takahashi, and K. Kimura, J. Forensic Sci. 39:362-372, 1994). The method and apparatus disclosed in this patent publication do not provide for a step to separate the red blood cells and platelets from the nucleated white blood cells. In addition, this patent publication primarily discloses the isolation of nucleic acids using microtiter plates and does not disclose a specific method to isolate RNA from larger volumes. In some analyses, total RNA is needed in larger quantities than can be isolated from microtiter plates.

Automated systems are currently being sold for the purpose of RNA isolation from whole blood, such as the Roche MagNA Pure LC System and the Qiagen BioRobot Universal System. However, these systems are generally meant for microliter quantities of sample and are based on a 96 well format. In some situations, the ability to isolate nucleic acids from larger sample sizes, such as a volume of 1 ml or more, is desirable. In addition, these systems are not compact and need a separate computer component. It would be advantageous to have a compact system that could be used in areas of the world where laboratories are not available. For example, a small RNA isolation system would allow the user to take fresh blood from a patient and insert it directly into the machine without much of a time lag for degradation of the RNA. In environments where sterility and refrigeration are limited, a biological isolation system that is immediate and compact is a great advantage.

Other systems, such as the ABI 6100 Nucleic Acid Prep Station (Large-volume format) can handle larger volumes of whole blood but are not fully automated and/or require cleaning of the system after use. For example, the ABI system, which can use a 3 ml sample size, requires several dilution steps of the whole blood prior to the automated steps and also needs several cleaning steps of the machine after the nucleic acid is isolated.

Consequently, the inventors have recognized that there exists a need in the art for a fully automated, compact, rapid biological molecule isolation system and method that can purify biological molecules from a relatively large volume of blood. For example, there exists a need for an RNA isolation system that is so fully automated that it does not require any manual pretreatment of the sample and does not require any cleaning between uses. In addition, the system should be able to separate white blood cells from red blood cells so that the isolated nucleic acid is high quality and will not inhibit subsequent analyses. Such a system should minimize, to the extent possible, the amount of skilled work that must be performed by users, to minimize or eliminate errors or variability between samples and testing facilities.

### SUMMARY OF THE INVENTION

The present invention addresses needs in the art identified by the inventors by providing a system for purification of biological molecules from samples. In exemplary embodiments, the system is a system for purifying in an automated fashion RNA from white blood cells present in whole blood samples. The system is an automated system that is rapid and reproducible, and provides high-quality highly purified molecules of interest. In general, the system comprises an instrument for automated purification of substances, computer software to control the instrument and purify the substance(s), one or more cartridges, packages, or containers for use with the instrument, and a method of purifying one or more biological materials. The system is an integrated system of multiple independent parts and features that can be designed to interconnect to provide the user the ability to purify numerous biological molecules from various different samples. The system can include use of core parts in conjunction with replaceable parts.

In one aspect, the invention provides an instrument for purifying one or more biological molecules of interest. In general, the instrument provides means for housing internal components, parts, elements, etc. of the instrument; means for moving at least one liquid composition from a storage means to a purification means; and at least one of the following: means for holding at least one of: means for purifying one or more biological materials, means for storing one or more liquid compositions, and means for containing one or more waste products of a purification process. In some embodiments, the instrument further comprises means for controlling the means for moving at least one liquid, means for controlling the movement of at least one liquid within the purification means, or both. In some embodiments, the instrument comprises an outer shell or case that houses one or more pumps for moving liquids from a reagent pack to a purification cartridge, and, optionally, a computing device capable of controlling the pump(s). In some embodiments, the instrument comprises one or more connectors that allow a reagent pack, a purification cartridge, or both, to be connected to the instrument and, preferably, to each other.

In another aspect, the invention provides means for purifying at least one biological molecule from a sample. In general, the purification means comprises one or more means for receiving and dispensing a liquid; one or more means for capturing cells; one or more means for binding nucleic acids; and one or more means for fluidly connecting the receiving, dispensing, capturing, and binding means. In some embodiments, the means for capturing cells preferentially captures nucleated cells. In some embodiments, the purification means is a purification cartridge comprising plastic having recesses disposed in one or more surfaces. In these embodiments, the recesses provide channels for connecting at least the following elements to one or more of the others: one or more inlet ports, one or more exit ports, one or more filters that capture cells, one or more filters that bind one or more nucleic acids and/or other biomolecules. In addition, one or more of the recesses may provide space for accommodating the filter(s).

In yet another aspect, the invention provides means for storing one or more liquid compositions. In general, the storage means comprises one or more independent means for storing one or more liquid compositions, each of which comprises or is fluidly connected to at least one means for conducting the respective liquid compositions out of the storage means. In embodiments, the storage means further comprises means for replacing volumes of liquid removed from the storage means to maintain a suitable pressure in the storage means. The storage means includes means for allowing fluid to exit the storage means. In embodiments, the storing means comprises a reagent pack comprising one or more containers that contain liquid compositions, each of which are connected to a tube, such as a piece of flexible, compressible tubing, that acts as a conduit from the container to one or more exit ports on the reagent pack. In some embodiments, the reagent pack comprises one or more containers that receive and contain waste products from a purification process.

In a further aspect, the invention provides means for receiving waste products from the purification means, the storage means, or both. In general, the waste receiving means comprises at least one means for receiving waste materials from the purification means, the storage means, or both; and means for containing the waste materials. In embodiments, the waste receiving means comprises at least one inlet port that is fluidly connected to at least one container by way of a tube or other conduit. In some embodiments, the container comprises a vent that allows a connection to the external environment, which can assist in maintaining suitable pressure in the container. In some embodiments, the waste receiving means is connected to a pressure generating means, which is responsible or is involved in movement of one or more liquids into the waste receiving means. In some embodiments, the pressure generating means is a pump that generates a vacuum in one or more containers, which causes or assists in drawing waste fluid into the container(s).

In yet a further aspect, the invention provides means for causing movement of fluid within and among the storing means, purification means, and waste receiving means. In general, the means comprises means for moving fluids within the system and means for regulating movement of the fluids. In embodiments, the means for causing movement of fluid comprises one or more pumps that mechanically force one or more fluids to enter and/or exit the storing means, the purification means, or the receiving means. Typically, the means for causing movement of fluid further comprises at least one controllable or adjustable valve that regulates movement of fluids through one or more conduits or filters. The adjustable valve can be located at any position along fluid flow lines, but is preferably located on the purification means. In some embodiments, all of the valves for regulating movement of fluids through the purification means (e.g., cartridge) are located on the purification means.

In an additional aspect, the invention provides means for controlling a process of purification of a biological substance from a sample. In general, the means for controlling a purification process comprises computer software (e.g., a program) that executes on a computing device to effect one or more steps in a purification process. The means for controlling typically comprises software that, when executed by a computing device, results in control of one or more mechanical devices of the system. For example, in embodiments, the software controls the timing and movement of one or more valves of the system, and controls the pumping action of a pump that moves liquids from the storage means to the purification means, with waste returning to a separate compartment, such as one in the storage means.

In yet an additional aspect, the invention provides computing means for controlling a process of purification of a biological substance from a sample. In general, the computing means comprises software and hardware for operating a computing device and executing software programs. The computing means can comprise commercially available hardware and software, and can use any of a number of standard components, computer languages, and the like.

In another aspect, the invention provides an automated method of purifying or isolating one or more substances from a sample. While not so limited, typically, the method is a method of purifying or isolating a substance from a sample comprising one or more biological molecules, such as a nucleic acid or protein. In general, the method comprises: exposing a sample comprising one or more substance of interest to a filtering means such that the substance is captured by the filtering means; releasing the substance of interest from the filtering means; and exposing the substance of interest to a binding means. In embodiments, substance of interest is a biological molecule found in a cell. In these embodiments, the step of exposing the sample to the filtering means results in binding of the cell to the filtering means, and the method further comprises lysing the cell to release the substance of interest. In the method, all of the steps are performed automatically by a machine, such as one controlled by a computer program. In other words, none of the steps of the method requires human interaction or human action, although certain optional steps (e.g., providing a sample) may include some human action. In embodiments, the method is a method of purifying or isolating RNA from white blood cells, such as those in samples comprising whole blood or cultured or transformed white blood cells.

### BRIEF DESCRIPTION OF THE DRAWINGS

The accompanying drawings, which constitute a part of this specification, illustrate several embodiments of the invention and, together with the written description, serve to explain various principles of the invention. It is to be understood that the drawings are not to be construed as a limitation on the scope or content of the invention.

FIG. 1 depicts an exemplary cartridge according to the invention. Panel A depicts the cartridge from a front perspective. Panel B depicts the cartridge from a rear perspective.

FIG. 2 depicts a cross-section of the exemplary cartridge of FIG. 1.

FIG. 3 depicts a pre-filtration unit according to an embodiment of the invention. Panel A depicts the unit from a proximal end, situated in a cartridge according to an embodiment of the invention. Panel B depicts a cross-sectional side view of the unit, situated in a cartridge according to an embodiment of the invention.

FIG. 4 depicts an exploded view of an exemplary system according to the invention.

FIG. 5 depicts a cross section of an additional embodiment of a cartridge according to the invention.

FIG. 6 depicts an embodiment of a rotary valve of the cartridge of FIG. 5, showing conduit connections between two pairs of ports (ports 1 and 2, and ports 6 and 7).

### DETAILED DESCRIPTION OF VARIOUS EMBODIMENTS OF THE INVENTION

Reference will now be made in detail to various exemplary embodiments of the invention. The following description is provided to give details on certain embodiments of the invention, and should not be understood as a limitation on the full scope of the invention.

Broadly speaking, the present invention provides an automated system for isolating or purifying a substance of interest from a sample containing it. In general, the system comprises mechanical equipment, containers for purification fluids, filtration cartridges, computer equipment, and computer software. The various components are configured to provide an automated method of purifying or isolating a target substance, such as a nucleic acid or protein. One feature of the invention is the adaptability that the system provides through the disposable or configurable nature of many of the elements of the system. For example, in preferred embodiments, a reagent pack comprising all of the compositions needed for purification of a target substance, such as RNA, is provided in a modular pack that can be connected or removed from the system independently of all other elements. Likewise, in preferred embodiments, a purification cartridge comprising filters for purifying a target substance is provided in a modular form that can be connected or removed from the system independently of all other elements. In a preferred embodiment, the automated system can be used for purification of RNA from white blood cells isolated from other components of a whole blood sample.

According to the present disclosure, all terms relating to the various aspects of the invention are used in accordance with their customary meanings in the art unless otherwise noted and specifically defined. For the purpose of providing a general context for certain terms, the following description is provided. The meanings of other words, terms, and phrases will be apparent from their standard meanings, the context of the sentence in which they are use, or by the descriptions of them provided below.

As used herein, the terms “isolating” and “purifying” are used interchangeably as terms that include the process of removing a substance from a composition of matter, such as removing RNA from a cell sample, and separating it from at least one other substance in the original sample. For example, isolating RNA can include separating it from other cellular material and other nucleic acids. Isolated RNA will be generally free from contamination by other nucleic acids and will generally have the capability of being reverse transcribed. Isolating or purifying does not require absolute isolation or purity. Rather, isolated substances, including RNA, are considered isolated or purified if separated from at least one other substance originally found present in a sample from which the substance is taken. In preferred embodiments, isolated or purified substances will generally be at least or about 30%, 40%, 50%, 60%, 70%, 80%, 85%, 90%, 95%, 98%, or 99% or more. Preferably, isolated RNA according to the invention will be at least 98% or at least 99% pure. It is to be understood that “isolating” and “purifying” refer to all substances, including RNA, DNA, protein, and other biochemical components of cells.

Where a value is stated herein, it is to be understood that, unless otherwise specifically noted, the value is not meant to be precisely limited to that particular value. Rather, it is meant to indicate the stated value and any statistically insignificant values surrounding it. As a general rule, unless otherwise noted or evident from the context of the disclosure or from the nature of experiments and their associated intrinsic variance, each value includes an inherent range of 5% above and below the stated value. At times, this concept is captured by use of the term “about”. However, the absence of the term “about” in reference to a number does not indicate that the value is meant to mean “precisely” or “exactly”. Rather, it is only when the terms “precisely” or “exactly” (or another term clearly indicating precision) are used is one to understand that a value is so limited. In such cases, the stated value will be defined by the normal rules of rounding based on significant digits recited. Thus, for example, recitation of the value “100” means any whole or fractional value between 95 and 104, whereas recitation of the value “exactly 100” means 99.5 to 100.4.

As used herein, the term “nucleic acid” includes polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). Thus, “nucleic acid” includes double- and single-stranded DNA, as well as double- and single-stranded RNA. The term “nucleic acid”, as used herein, also includes polymers of ribonucleosides or deoxyribonucleosides that are covalently bonded, typically by phosphodiester linkages between subunits, but in some cases by phosphorothioates, methylphosphonates, and the like. Such nucleic acids include, but are not limited to, gDNA; hnRNA; mRNA; noncoding RNA (ncRNA), including but not limited to rRNA, tRNA, miRNA (micro RNA), siRNA (small interfering RNA), snORNA (small nucleolar RNA), snRNA (small nuclear RNA), and stRNA (small temporal RNA); fragmented nucleic acid; nucleic acid obtained from subcellular organelles, such as mitochondria or chloroplasts; and nucleic acid obtained from microorganisms, parasites, or DNA or RNA viruses that might be present in a biological sample. Synthetic nucleic acid sequences, that might or might not include nucleotide analogs, that are added or “spiked” into a biological sample are also within the scope of the invention. Reference to one strand of a nucleic acid inherently includes a reference to a complementary strand.

A “protein”, “polypeptide”, or “peptide” according to the invention is a molecule comprising at least one amide bond linking two or more amino acid residues together. Although used interchangeable, in general, a peptide is a relatively short (e.g., 2-10 amino acid residues in length) molecule, a protein is a relatively long (e.g., 100 or more residues in length) molecule, and a polypeptide is an intermediate-length molecule (e.g., 10-100 residues). However, it is to be noted that, unless specifically defined by a chain length, the terms peptide, polypeptide, and protein are used interchangeably. Those of skill in the art will immediately recognize that these molecules can range from two residues to hundreds or more residues in length. It is thus unnecessary for a specific recitation of each and every number from two to many hundreds or greater to be made herein in order for those of skill in the art to understand that each specific value/number is encompassed and envisioned by the invention. Accordingly, each value will not be specifically recited herein, although each value is to be understood as recited intrinsically by this disclosure. This concept is also applied to nucleic acid chain lengths in the context of the discussion above and throughout this document.

As used herein, the terms “solid phase substrate” and “solid support” are used interchangeably, and include solid phase materials, also referred to as solid phases or solid phase supports, that are capable of binding substances of interest. Exemplary substances discussed herein include nucleic acids, proteins, and other biomolecules that are present in or are released from a biological sample. Numerous such solid phase substrates are known in the art, and the identity of each need not be disclosed herein. Exemplary solid phase substrates include variety of materials that are capable of binding nucleic acids under suitable conditions. They include, but are not limited to, compounds comprising silica, including but not limited to, silica particles, silicon dioxide, diatomaceous earth, glass, alkylsilica, aluminum silicate, and borosilicate; nitrocellulose; polymers; diazotized paper; hydroxyapatite (also referred to as hydroxylapatite); nylon; metal oxides; zirconia; alumina; diethylaminoethyl- and triethylaminoethyl-derivatized supports (e.g., Chromegabond SAX, LiChrosorb-AN, Nucleosil SB, Partisil SAX, RSL Anion, Vydac TP Anion, Zorbax SAX, Nucleosil Nme2, Aminex A-series, Chromex, and Hamilton HA lonex SB, DEAE Sepharose, QAE Sepharose); hydrophobic chromatography resins (such as phenyl- or octyl Sepharose); “affinity based” purification resins; and the like. The terms solid phase and its equivalents are not intended to imply any limitation regarding form. Thus, the term solid phase encompasses appropriate materials that are porous or non-porous; permeable or impermeable; including but not limited to membranes, filters, sheets, particles, beads, including magnetic beads, gels, powders, fibers, and the like. Solid phase supports can include a single membrane, filter, bead, etc. or two or more of these forms. Likewise, solid phase supports may comprise two or more different forms combined into a single functional unit.

Thus, the “solid phase substrate” can be a filter or a filter membrane. The term “filter membrane” or “matrix” as used herein includes a porous material or filter media formed, either fully or partly from glass, silica or quartz, including their fibers or derivatives thereof, but is not limited to such materials. Other materials from which the filter membrane can be composed also include cellulose-based (nitrocellulose or carboxymethylcellulose papers), hydrophilic polymers including synthetic hydrophilic polymers (e.g., polyester, polyamide, carbohydrate polymers), polytetrafluoroethylene, porous ceramics, nylon, polysulfone, polyethersulfone, polycarbonate or polyacrylate, as well as acrylic acid copolymers, polyurethane, polyamide, polyvinyl chloride, polyfluorocarbonate, polybutylene terephthalate, polytetrafluoroethylene, polyvinylidene fluoride, polyvinylidene difluoride, polyethylene-tetrafluoroethylene copolymer, polyethylene-chlorotrifluoroethylene copolymer, or polyphenylene sulfide. For immobilization of nucleic acids onto the membranes or filters, there may be used salts of mineral acids and alkali or alkaline earth metals, salts of alkali or alkaline earth metals and monobasic, polybasic or polyfunctional organic acids, hydroxyl derivatives of hydrocarbons, or chaotropic agents, among other things.

As used herein, “sample” includes anything containing or presumed to contain a substance of interest. It thus may be a composition of matter containing nucleic acid, protein, or another biomolecule of interest. The term “sample” thus includes a sample comprising nucleic acid (genomic DNA, cDNA, RNA, protein, other cellular molecules, etc.), one or more cells, one or more organisms, one or more tissues, and one or more fluids, which may comprise one or more dissolved, suspended, or particulate solids. Exemplary compositions and substances include, but are not limited to, external secretions of the skin, respiratory, intestinal and genitourinary tracts; tumors; samples of in vitro cell culture constituents; natural isolates (such as drinking water, seawater, solid materials); microbial specimens; and objects or specimens that have been “marked” with nucleic acid tracer molecules. Exemplary samples include whole blood or compositions comprising whole blood, from any animal, including, but not limited to humans, companion animals (pets), and farm or agricultural animals.

The term “sample” is thus used in a broad sense and is intended to include a variety of biological sources that contain nucleic acids and/or protein and/or a biomolecule of interest. Exemplary biological samples include, but are not limited to, whole blood, plasma, serum, white blood cells, red blood cells, buffy coat, swabs (including but not limited to buccal swabs, throat swabs, vaginal swabs, urethral swabs, cervical swabs, rectal swabs, lesion swabs, abscess swabs, nasopharyngeal swabs, and the like), urine, stool, sputum, tears, saliva, semen, lymphatic fluid, amniotic fluid, spinal or cerebrospinal fluid, peritoneal effusions, pleural effusions, fluid from cysts, synovial fluid, vitreous humor, aqueous humor, bursa fluid, eye washes, eye aspirates, pulmonary lavage, lung aspirates, and organs and tissues, including but not limited to, liver, spleen, kidney, lung, intestine, brain, heart, muscle, pancreas, and the like. The skilled artisan will appreciate that lysates, extracts, or material obtained from any of the above exemplary biological samples are also within the scope of the invention. Tissue culture cells, including explanted material, primary cells, secondary cell lines, and the like, as well as lysates, extracts, or materials obtained from any cells, are also within the meaning of the term biological sample as used herein. Microorganisms and viruses that may be present on or in a biological sample are also within the scope of the invention. Materials obtained from clinical or forensic settings that contain nucleic acids are also within the intended meaning of the term biological sample.

As used herein, the term “biological sample” or “sample” also refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid, and semen). The term “biological sample” further refers to a homogenate, lysate, or extract prepared from a whole organism or a subset of its tissues, cells or component parts, or a fraction or portion thereof. Furthermore, “biological sample” refers to a medium, such as a nutrient broth or gel in which an organism has been propagated, which contains cellular components, such as proteins or nucleic acid molecules. The terms “isolated” and “purified” mean that the biological molecule or cell is separated from other substances in the sample. These substances may include different types of molecules or cells as compared to the molecule or cell that is being isolated. For example, nucleic acids may be isolated from other biological molecules such as proteins, carbohydrates, and lipids, and any other molecule found in cells. In another example, white blood cells may be separated from red blood cells. Substances may also refer to the same type of molecule or cell as compared to the molecule or cell that is being isolated. For example, one specific protein may be isolated from other proteins or one kind of nucleic acid may be purified away from other types of nucleic acids. The biological molecule or cell may also be separated from other substances such as debris from lysed or sheared cells or tissue components such as cellular organelles and connective tissue. Substances may also include whatever buffer or liquid the cells or tissue were in such as a lysis buffer or media for growing cultured cells. The biological molecule or cell can be partially purified with the methods of this invention such that it is partially separated or partially purified from some of the other substances in the sample. The biological molecule or cell can be mostly purified such that it is more than 80% pure. It can also be pure or almost pure such that at least 95%, such as 98%, 99%, 99.5%, or greater of the biological material in the final elution comprises the biological molecule or cell of interest. Of course, any level of isolation or purity is envisioned by this invention, from 1% to 100%, and all of the particular values within this range, including fractions thereof, are contemplated, and it is to be understood that those of skill in the art will immediately recognize each particular value within the range without each particular value needing to be recited specifically herein. As used herein, isolated and purified are used interchangeably. In a preferred embodiment, isolation occurs as a fully automated method, where the user inserts a sample into the system and takes out the isolated biological molecules or cells from the instrument.

Thus, as used herein, the term “biological molecule” refers to any molecule found within a cell or produced by a living organism, including viruses. This may include, but is not limited to, nucleic acids, proteins, carbohydrates, and lipids. In preferred embodiments, a biological molecule refers to a nucleic acid or a protein, and most preferably to a nucleic acid. As used herein, a “cell” refers to the smallest structural unit of an organism that is capable of independent functioning and is comprised of cytoplasm and various organelles surrounded by a cell membrane. This may include, but is not limited to, cells that function independently such as bacteria and protists, or cells that live within a larger organism such as leukocytes and erythrocytes. As defined herein, a cell may not have a nucleus, such as a mature human red blood cell. “Blood cell” refers to cells found in the blood such as erythrocytes, leukocytes, and platelets.

A biological molecule or cell can be isolated from various samples such as tissues of all kinds, cultured cells, body fluids, whole blood, blood serum, plasma, urine, feces, microorganisms, viruses, plants, and mixtures comprising nucleic acids following enzyme reactions. Examples of tissues include tissue from invertebrates, such as insects and mollusks, vertebrates such as fish, amphibians, reptiles, birds, and mammals such as humans, rats, dogs, cats and mice. Cultured cells can be from procaryotes comprising the archaebacterial domain or the eubacterial domain. Cultured cells can also be from procaryotes comprising a cell wall such as bacteria, blue-green algae, actinomycetes, and from procaryotes without a cell wall such as mycoplasma. Cultured cells can also be from eucaryotes such as plants, animals, fungi, algae, slime molds and protozoa. Blood samples include blood taken directly from an organism or blood that has been filtered in some way to remove some elements such as serum or plasma. Blood samples also include blood components, such as a sample that comprises white blood cells and/or red blood cells. Samples for the methods of the invention can be used fresh, such as blood samples that have recently been taken from an organism, or can be used after being stored in a refrigerator or freezer for an extended period of time, such as a cryopreserved sample. Samples may be taken from the environment, such as from a body of water or from soil. Although the method is envisioned in many cases to be fully automated, there may be samples that require some pretreatment. For example, lytic enzymes may be added to degrade cell walls. As another example, a cell culture may be centrifuged to reduce the volume of the sample. Also, tissue may be chemically or physically broken down, such as by using enzymes or a grinding apparatus.

The term “buffer” includes aqueous solutions or compositions that resist radical changes in pH when acids or bases are added to the solution or composition. This resistance to pH change is due to the buffering properties of such solutions, and may be a function of one or more specific compounds included in the aqueous composition. Thus, solutions or compositions exhibiting buffering activity are referred to as buffers or buffer solutions. Buffers generally do not have an unlimited ability to maintain the pH of a solution or composition. Rather, they are typically able to maintain the pH within certain ranges, for example between pH 5 and pH 7. Typically, buffers are able to maintain the pH within one log above and below their pKa. Those of skill in the art are well aware of the numerous buffers available for buffering compositions, and all such buffers and their use need not be detailed herein. Exemplary buffers include, but are not limited to, sodium carbonate/bicarbonate, MES ([2-(N-Morphilino)ethanesulfonic acid]), ADA (N-2-Acetamido-2-iminodiacet-ic acid), Tris ([tris (Hydroxymethyl)aminomethane]; also known as Trizma); Bis-Tris; ACES; PIPES; MOPS; and the like. Buffers and buffer solutions are typically made from buffer salts. Thus, for example but not as a limitation, to make a MES buffer one would use 2-(N-Morphilino)ethanesulfonic acid (or salts thereof); to make Tris buffer one would use Trizma base (or salts thereof) or Trizma HCl (or salts thereof), as appropriate; and so forth. Buffer solutions and buffer salts are commercially available from numerous sources, such as Sigma-Aldrich (St. Louis, Mo.), Fluka (Milwaukee, Wis.), and CALBIOCHEM (La Jolla, Calif.).

The term “chaotrope” or “chaotropic agent” or “chaotropic salt”, as used herein, includes substances that cause disorder in a protein or nucleic acid by, for example, but not limited to, altering the secondary, tertiary, or quaternary structure of a protein or a nucleic acid while leaving the primary structure intact. Exemplary chaotropes include, but are not limited to, guanidine hydrochloride (GuHCl), guanidine thiocyanate (GuSCN), potassium thiocyanate (KSCN), sodium iodide, sodium perchlorate, urea, and the like. A typical anionic chaotropic series, shown in order of decreasing chaotropic strength, includes: CCl₃COO>CNS>CF₃COO>ClO₄>F>CH₃COO>Br, Cl, and CHO₂. Descriptions of chaotropes and chaotropic salts can be found in, among other places, U.S. patent application publication number 2002/0177139 and U.S. Pat. No. 5,234,809, the disclosures of which are hereby incorporated herein by reference. Exemplary chaotropes include some non-ionic detergents.

Turning now to the details of certain embodiments of the invention, in a first aspect of the invention, an instrument for purifying one or more substances of interest is provided. The instrument is designed to perform various functions in the process of purifying the substance of interest, which may be any substance that has a property of interest to a person practicing the invention. The substance thus may be a synthetic organic or inorganic chemical (e.g., a drug or other bio-acting agent), a biological molecule (i.e., a molecule produced by a living organism, such as, but not limited to, a drug or other bio-acting agent, a nucleic acid, and a protein; also referred to herein as a biomolecule), or another substance. In exemplary embodiments, the substance is a biological molecule of interest, such as DNA, RNA, or protein.

In general, the instrument comprises means for housing one or more elements that function in a process for purifying a substance. In embodiments, the housing means is a container for internal parts, such as an outer housing, shell, cabinet, or box. The housing means may be of any size and shape that is suitable for housing desired parts. Thus, it may be relatively small (e.g., portable) and suitable for placement on a laboratory benchtop or cart, or it may be relatively large and designed to be a stationary piece of equipment. The housing means may be fabricated from any suitable material or combinations of materials, including, but not limited to, metals, plastics (including, but not limited to, thermoplastics and thermosetting resins), wood, glass, and rubber (natural or synthetic). In its basic form, it includes an outer surface that is substantially exposed to the external environment, and an inner surface that is substantially exposed to the inside of the housing. Either or both of the external surface and the internal surface may comprise means for securing the housing to one or more other objects, to internal components housed in the housing, or to itself (e.g., to provide rigidity and/or stability to the housing). The housing means can perform multiple functions, including, but not limited to, one or more of the following: a shell for protection of internal parts of the instrument, a container for internal components that can reduce noise created by those components, and a design or overall look that provides an aesthetic appearance for users.

The outer surface of the housing means can comprise one or more means for attaching other objects to the housing means. These attachment means may comprise any suitable structures for attaching one object to another. Thus, the attachment means may be a hole into which a screw, bolt, pin, rod, etc. may be inserted to attach an object. Likewise, the attachment means may be a bracket, flange, etc. to which an object may be attached. Other non-limiting attachment means includes hook-and-loop fasteners. Attachment may be in any suitable way, both permanently or removably. Thus, for example, an object may be bolted or screwed to the housing means and removed at a later time for replacement/repair. Likewise, an object may be attached by way of one or more friction-fit couplings, such as spring clamps, which can securely hold the object while permitting release of the object when desired. Release may be possible by simple human strength or through use of a tool.

In some embodiments, the housing means comprises one or more means for attaching elements of the system of the invention to the instrument. For example, in some embodiments, the housing means includes one or more recesses in the outer surface of the housing means, which are designed to accommodate and releasably secure a reagent pack (see below), a purification cartridge (see below), or both. Alternatively, the housing means may comprise one or more clamps for releasably securing a reagent pack, a purification cartridge, or both. Yet again, the surface may comprise one or more invaginations, holes, or the like for receiving pins, rods, or the like, on a reagent pack, a purification cartridge, or both, wherein alignment of the pins and holes releasably secures the pack and/or cartridge to the housing. For example, the instrument may comprise one or a series of peristaltic pumps that receive compressible tubing of the reagent pack or attached to the reagent pack. Each tube may have a connector at each end, which are fixed in place in the instrument housing by a bracket or other attachment means. The tubes may be affixed to the housing means or pump head by way of grooves or channels formed in the reagent pack, which may also provide pressure to seat and retain the tubes against the pump head. The tubes may have a male end that is designed to couple with and insert into female receptacles on the containers of the reagent pack. Likewise, the tubing may have female receptacles, such as those defined at a surface by O-rings or other seals, for receiving male connectors of the purification cartridge. These connectors may assist in retaining the reagent pack, the cartridge, or both, to the instrument.

As a general matter, in embodiments, the instrument housing means comprises at least one surface that mates with a reagent pack, a purification cartridge, or both. As will be detailed below, this mating allows for interaction of one or more components housed in the housing means with the reagent pack, purification cartridge, or both. Thus, in embodiments, at least one surface of the housing is designed to accommodate and interact with a removable element of the system. Typically, the housing means will comprise a particular design for a particular use, and the reagent pack, purification cartridge, or both will be designed to successfully engage with the housing means.

Where the housing means comprises a surface that contacts and interacts with a reagent pack and/or purification cartridge, the surface may comprise a hole, port, or other opening that allows at least a part of a component housed in the interior of the housing means to be exposed to the exterior of the housing means. For example, in embodiments, the housing means comprises one or more openings through which a portion of one or more peristaltic pumps extends. The exposed portion of the peristaltic pump is configured such that it may contact a portion of a reagent pack or tubing connected to the reagent pack when the reagent pack is placed in contact with the housing surface. In this way, the pump may contact one or more pieces of tubing exposed on the reagent pack or provided separately, causing pumping of the fluid in the tube upon movement of the pump. Of course, in these embodiments, the opening in the outer surface of the housing means will be designed to suitably accommodate the portion of the pump that will form a portion of the housing surface, and the outer surface and/or inner surface of the housing means will be fabricated to include attachments to secure the pump in the appropriate place.

It is to be noted that there may be one or more pumps, such as peristaltic pumps, within the housing or partially disposed on a surface of the housing, or there may be a single pump with multiple heads. The distinction is not critical so long as the pump(s) may accommodate one or more tubes for pumping of one or more fluids from a reagent pack to a cartridge. For example, a peristaltic pump can be used to force multiple liquids and air through tubing comprising part of a reagent pack, out exit ports of the reagent pack, and into entry ports of a purification cartridge. A single pump head may be used to pump multiple fluids; ports not requiring fluids at a given time may be effectively closed by valving provided on the cartridge (see below).

The outer surface of the housing means can also comprise an interface for interaction of users with internal components housed within the housing means or with other components of the system of the invention. More specifically, the housing means can include a surface that includes an interface, such as a keyboard, touch-screen, or the like, that allows users to program or control the instrument and peripherals (e.g., a computer running a computer program) for purification of a substance of interest. The interface may be presented in any suitable fashion and may include controls that are configured in any suitably way. Typically, the interface will provide a user the ability to create a purification scheme or protocol or to select a pre-designed scheme or protocol, initiate performance of the protocol, and terminate the protocol. Various other features may be provided, including the ability to uniquely identify and label various samples (e.g., barcoding of the sample and cartridge), to correlate a particular sample and/or reagent pack with a particular purification run or purification scheme, to analyze purification scheme parameters, and the like. In essence, the interface can allow users to obtain any and all information available relating to a particular purification run, the amount of information being only limited by the software implementing the purification scheme, the quality of the sample, and the configuration of detections (if any) present on the machine. Of course, the interface can include one or more panels, screens, etc. for display of graphical or textual information to the user. In some embodiments, the interface can comprise means for producing printed information on paper, although in other embodiments, the means for producing printed information is provided at a different location on the instrument or as a separate, stand-alone device.

As mentioned above, the housing means houses one or more internal components, devices, apparatuses, etc. In general, the housing means houses at least one pump that is used to pump fluids through a purification cartridge for purification of a substance of interest and/or removal of waste products. The pump is not limited in its size, shape, or any particular feature, as long as it is suitable for pumping at least one fluid through a purification cartridge according to the invention. Typically, the pump is a mechanical pump, such as a peristaltic pump, which can be used to pump fluids through flexible tubing. As discussed in detail below, the present invention provides the ability to pump multiple different fluids during the process of purification of a substance. Accordingly, the instrument of the present invention can comprise one or more pumps for pumping these fluids. Although not required, it is preferred that all of the pumps of the system be housed within the housing means. In one non-limiting example, one or more peristaltic pumps are provided for pumping one or more liquids from a reagent pack to a purification cartridge. In embodiments, this peristaltic pump also serves to pump air or another gas when needed. In embodiments, at least one separate pump is provided to pump air or another gas into and through the purification cartridge. Further, in some embodiments, a pump is provided to create a negative pressure at one or more outlet/exit ports of the purification cartridge. In embodiments, a single motor may serve two or more pumps. For example, a single motor may run an air pressure pump and an air vacuum pump.

With regard to the various possible pump configurations, it is to be noted that the use of two pumping mechanisms to achieve fluid flow within the system can provide an advantage over other configurations. More specifically, as discussed below, one advantageous feature of an embodiment of the present methods and systems is the ability to move fluids through a series of conduits and chambers at a pressure that is relatively moderate (thus reducing the likelihood of catastrophic failure of parts) and that shows a substantial pressure drop across membranes or other permeable barriers. By providing a positive pressure behind the fluid to be moved (i.e., a “pushing” force) and concurrently providing a negative pressure ahead of the fluid to be moved (i.e., a “pulling” force), the total pressure of the system can be maintained at a relatively low level while achieving a high flow rate and a high pressure differential from the front of the fluid to the back. This high pressure differential has been found to be advantageous in the purification methods of the present invention.

The system of the present invention is an automated system. Accordingly, the action of each pump housed in the housing means can be controlled by a computer. That is, although it is possible to run all of the needed pumps throughout the purification process and control fluids to be pumped by controlling valves for each fluid, it is also possible to control pumping of fluids by controlling the action of the pump (i.e., by turning pumps on and off as needed or regulating the speed by regulating power to the pump). Each of these controlling actions can be effected by a computer connected to the pumps and valves of the system.

The instrument may comprise two or more means for moving fluids, such as two or more pumps. Typically, at least one pump is a peristaltic pump that moves fluids by compressing flexible tubing holding the fluid. It is to be noted that, in contrast to other systems known in the art, which pump fluids through an instrument, the system of the present invention allows for pumping of fluids without the fluids entering the instrument itself. That is, the instrument of the present invention is configured such that the pump head of a peristaltic pump is present at a surface of the instrument housing. The pump head may thus engage fluid-filled tubing (e.g., tubing containing a liquid) and pump the fluid through the tubing without the tubing or the fluid entering the instrument itself. In this way, cleaning of the instrument is eliminated, and cross-contamination of samples from one batch to the next as a result of inadequate cleaning is avoided. It is to be noted that, in embodiments, the peristaltic pump is provided with tubing, which is then connected to a reagent pack and a purification cartridge. However, in these embodiments, the tubing is disposable and is not to be considered part of the pump (or instrument), but rather as a consumable item used in conjunction with the pump and instrument. Further, although air and gases can be considered fluids, and can be pumped through the instrument using one or more pumps, there is no need to clean any instruments or tubing after pumping of air or other gases.

In addition to the one or more pumps for pumping fluids through a purification cartridge, the instrument may comprise one or more pumps for pulling fluids through a purification cartridge. More specifically, as will be discussed in detail below, the purification cartridge comprises multiple small-diameter channels through which various fluids must travel. The amount of pressure required to successfully pump fluids through some of the channel pathways can become high. To reduce the amount of force required to push fluids through the purification cartridge channels, in embodiments, means for pulling fluids through the channels is also employed. For example, a vacuum pump may be housed in the housing means and may be connected to a waste reservoir, which in turn is connected to one or more outlet ports of the purification cartridge. Alternatively for example, a vacuum pump may be directly connected to one or more outlet ports of a cartridge, and cause a vacuum to be generated in the conduits and compartments of the cartridge. Engaging the vacuum pump causes a negative pressure on the exit port, which is transmitted through the channels and results in a “pulling” force, which augments the “pushing” force of the peristaltic pump, reducing the total pressure needed to “push” the fluid through the purification cartridge. Where one or more means for creating a vacuum are used, the respective pressures provided by the pumps of the system can be coordinated to provide suitable pressures at the leading and trailing edges of the fluid of interest to ensure sample stability and maintain substantially equal fluid flow characteristics throughout the fluid.

In accordance with the disclosure above, the instrument of the invention comprises means for moving at least one fluid, such as a liquid composition, from a storage means, such as a reagent pack, to a purification means, such as a purification cartridge comprising filters for separating a substance of interest from other substances. In addition to the means for housing internal components (e.g., an outer shell), the instrument can further comprise at least one of the following: means for holding a reagent pack, means for holding a purification cartridge, and means for holding a waste product pack.

The instrument may comprise means for attaching and/or securing means for containing waste material from the purification process of the invention. More specifically, the process of purifying a substance results in waste products being formed. Typically, the waste products are liquids that have been passed through the purification means. Non-limiting examples include filtered sample, wash buffers, binding buffers, elution buffers, and water. To assist in maintaining a clean laboratory environment and potentially to satisfy local, state, or federal requirements, some or all of the waste products can be collected in a waste collection means, and discarded when and where appropriate. As a general matter, the waste collection means can be any suitable container for receiving and containing waste substances, and in particular, liquid (e.g., aqueous, organic solvent) compositions. In embodiments, the waste collection means can be a container (e.g., bag, bottle, jar) that is fluidly connected to one or more exit ports of a purification cartridge, and is capable of receiving fluids exiting the exit port. The containers of the waste containment means can be rigid or collapsible/expandible and can be provided in a vacuum-sealed state, and can expand as needed to accommodate inflow of waste. Alternatively or in addition, the waste container can be vented to allow for pressure changes as waste flows in. In embodiments where a vacuum pump is used to facilitate flow of fluids through the purification cartridge, the waste containment means may comprise a vent that is fluidly connected to a vacuum pump, whereby vacuum created by the pump is applied to the waste container and the connector (e.g., tube) from the container to the inlet port of the containment means, when connected to an exit port of the purification cartridge, allows the vacuum to be applied to one or more channels of the purification cartridge.

In some embodiments, the instrument comprises means for controlling the movement of fluid within the system. The means for controlling fluid flow can be a single element or may comprise a collection of elements that work in concert to control fluid flow. In its basic form, the means for controlling fluid flow comprises a computing device that runs software that controls the activity of the pump(s) and valve(s) of the system. In various configurations, the means for controlling fluid flow comprises one or more valves disposed on a purification cartridge, which are controlled by a computing device running software that actuates the mechanical motions of the valves. The valves may be any type of valve known in the art, including mechanical valves that are actuated by physical movement of one or more parts by an actuator, or by electromagnetic forces or other natural phenomena (e.g., magnetically actuated valves) that cause the valve to move to a desired position (e.g., fully opened, fully closed, partially open). One advantageous feature of embodiments of the invention is placement of all valves on the purification cartridge, allowing for close control of fluids and reduction in dead volumes. A further non-limiting advantage of embodiments is the use of rotary valves that allow for selection of multiple channels/conduits for flow of multiple different fluids to multiple different other channels/conduits, and the ability to close or block multiple conduits with a single valve.

The system of the invention thus may comprise computer hardware and software to control movement of fluids, for example by controlling the action of pumps and valves, and to control the overall implementation of a purification scheme. The computer hardware and software is preferably comprised in the instrument of the invention. However, in embodiments, it is housed in a separate unit and connected to the instrument. In this regard, connection can be either physical connection (e.g., by way of cables, cords, etc.) or by way of electromagnetic radiation (e.g., by way of infrared, microwave, radio, etc. communication). Due to the versatility of implementation of computer systems, the computing hardware and software of the present system may be implemented as a single physical or functional unit or as discrete units. The invention thus provides an automated system useful for automating the isolation methods described herein. The system is useful in embodiments for isolating a biomolecule from a sample, in particular a cell sample. In one embodiment, the system is useful for isolating a biomolecule from a blood sample. The computer hardware and software may be comprised of commercially available components and/or written in any known language that may be compiled and run on a commercially available machine. The practitioner is free to chose the hardware and software combinations that suit a particular need or desire.

In general, embodiments of a system of the invention, which comprises an instrument as described above and is designed for performing the methods of the invention, comprises at least one removable cartridge comprising at least a first solid phase substrate disposed in a contained reservoir or chamber, a second solid phase substrate disposed in a contained reservoir or chamber, and an optional collection chamber (also referred to herein as a substance collection port). A cartridge according to the invention may contain the solid phase substrate(s) and one or more passageways through which a sample comprising a substance of interest and solutions containing substances and/or reagents for purification of substances can flow. A cartridge according to the invention also may contain one or more valves on the cartridge, such as one or more rotary valves on the cartridge, which may be the only valves present in the system for direct control of movement (i.e., contact) of fluids through the system. According to the invention, the valves are independently controllable by computer-control means, and can independently address more than two pathways per valve (i.e., the valves are not simple on-off valves, but allow for selection of three or more different options. Furthermore, according to the invention, one or more mixing chambers may be provided on the cartridge for mixing of substances of interest with reagents useful in its purification, where the substance and the material with which it is to be mixed are both in a liquid composition. Buffers and other liquid compositions that can be used to isolate the substance (e.g., biomolecule or biomolecules) of interest are contained in a reagent pack, which can also be a removable component of the system. A cartridge of the invention is useful for isolating any one of RNA or DNA or protein. In exemplary embodiments, it is useful for purifying RNA from white blood cells of a sample comprising whole blood. A cartridge of the invention may have one or more ports or collection chambers to collect one or more isolated materials. An instrument is also an integrated part of the system, and it provides the force (e.g., positive air pressure and/or vacuum) to drive liquids from the reagent pack into the cartridge and then through the cartridge for purification of the substance of interest.

In various embodiments, the system can be used to: purify a single material from a sample, for example RNA; to isolate any combination of RNA, DNA, protein, and any other biomolecule of interest either simultaneously or sequentially; to perform the isolation methods described herein in a uniform manner; and to perform the isolation methods described herein in a uniform manner, wherein the starting material is a cell sample, for example blood from one or more individuals. It is to be noted that purification may comprise binding of some or all contaminating materials while allowing the substance of interest to remain in solution, or may comprise binding of the substance of interest to one or more solid substrates.

In another aspect of the invention, means for purifying at least one substance of interest is provided. As discussed above, the substance may be any substance of interest. In exemplary embodiments, the substance is a biomolecule, such as a nucleic acid or protein. In general, the means for purifying at least one substance comprises: at least one means for receiving and dispensing a fluid (e.g., a liquid sample, such as blood); at least one means for retaining large substances (e.g., capturing cells); at least one means for capturing the substance of interest (e.g., a nucleic acid), and at least one means for fluidly connecting the receiving, dispensing, retaining, and capturing means, wherein the purifying means is configured such that the substance of interest is conveyed by fluid motion in a direction that exposes it to the above-mentioned means in the order in which they are described. In exemplary embodiments, the means for purifying a substance is configured for purifying nucleic acids from blood samples, and in particular for purifying RNA from white blood cells. In these embodiments, the purification means comprises: means for receiving a blood sample; means for conducting all or part of the blood sample to a means for retaining at least some of the cells in the sample; means for lysing the retained cells to release nucleic acids; means for retaining some, essentially all, or all of the DNA released from the cells upon lysis; and means for capturing RNA released upon lysis of the cells. For ease of reference, the purification means may be referred to herein as a purification cartridge.

In general, the purification cartridge comprises a body having disposed therein one or more channels and reservoirs for movement of fluids and containment of solid supports. The purification cartridge can be made of any suitable material or combination of materials. For example, it can be made from any of a number of plastic materials, such as plexiglass, polystyrene, nylon-66, or polycarbonate. Preferably, at least one surface of the cartridge is transparent or translucent to assist users in manually determining if fluids are flowing through the cartridge as intended. The cartridge may comprise one or more detection zones, such as transparent windows, for detecting (e.g., optically) or otherwise measuring one or more substances of interest.

The body of the purification cartridge provides the main physical support for the cartridge and its components. The cartridge is typically formed in two parts, a block (or shell) and a face (or cover), each having mating surfaces for the other. One or both of the surfaces are treated to form one or more channels and reservoirs. The treatment can be any process that results in suitably sized and shaped formations being created. For example, the cartridge body block may be etched, gouged, drilled, chemically dissolved, routed, or pre-cast to have the desired channels and reservoirs. Certain channels will be disposed in the cartridge body in a manner that allows for fluid communication with the external environment (i.e. exit and entrance ports will be created). The block and face may be made from any suitable material, such as a plastic material. For example, the face may be made from a tape or ribbon of plastic, or may be a thicker substance that has intrinsic rigidity. After creating the channels and reservoirs, the two parts of the cartridge body can be fused together by a suitable process, such as by application of one or more adhesives, by heat welding, sonication welding, or by dissolving and re-forming one or more portions of one or more of the parts to cause adhesion of the face to the block. Preferably, fusion is permanent and creates a fluid-tight seal along at least the channels and reservoirs.

The size and shape of the purification cartridge is not critical, but instead is designed in conjunction with the instrument and reagent pack to provide an integrated system for purification of substances. In embodiments, it has at least one surface that is square to rectangular in shape, which may have square or rounded edges. In some embodiments, the cartridge is approximately five inches to approximately twelve inches in length and/or width, and approximately one to approximately three inches in height/depth.

The purification cartridge comprises, on one or more outside surfaces of the body, attachment means for attaching the cartridge to the instrument of the invention. The means for attaching the cartridge comprises any suitable structure that can be used to physically attach the cartridge to the instrument. Where appropriate, the means complements the attachment means of the instrument. For example, where the instrument comprises spring clips for retaining the cartridge on the instrument, the cartridge can be designed such that one dimension is a size that is suitable for secure connection and retention by the spring clip. Alternatively, where the instrument comprises one or more holes in its surface, the cartridge can comprise one or more pins that can align with and insert into the holes. In addition, where the instrument comprises a bracket, the cartridge can comprise a complementary structure that fits into and/or attaches to the bracket.

The purification cartridge can also comprise, on one or more outside surfaces, attachment means for attaching the cartridge to a reagent pack. As with other attachment means discussed herein, the attachment means may comprise any suitable structure for attaching, either permanently or, preferably, releasably, the purification cartridge and reagent pack. Attachment of the purification cartridge to the reagent pack should cause ports in each to align such that fluid in one can flow into the other. In embodiments, port alignment and physical contact is adequate to attach the cartridge to the reagent pack.

In addition, in embodiments, the purification cartridge further comprises attachment means for attaching to a waste receiving means. Any suitable structure for connecting these two elements can be used. Attachment of the purification cartridge to the waste receiving means should cause ports in each to align such that fluid in one can flow into the other.

The purification cartridge comprises channels, valves, and solid supports for purification of substances of interest. The cartridge can be used in a manner that provides a purified product in a reservoir for removal, or provide a purified product bound to a solid support. The cartridge can be designed for single use (i.e., as a disposable element), or can be designed for multiple uses. Indeed, in embodiments where the cartridge block and face are fused in a manner that allows for easy removal of the face, for example when using relatively weak adhesive to hold the face to the block, the cartridge can be opened and cleaned, and solid supports removed and replaced.

Within the exterior surfaces (i.e., disposed within the body of the cartridge), the purification cartridge comprises one or more entrance or exit ports, one or more inlet ports, one or more valves, a pre-filter, and a filter, and, optionally, a target substance collection port. These elements are connected among each other by way of one or more conduits. As discussed above, these elements may be fabricated into the cartridge by etching, carving, cutting, drilling, molding, etc. the body of the cartridge to achieve the desired size, shape, and interconnectivity of each element. The size of the conduits and ports can be adjusted to fit the needs of a particular purification scheme. However, as a general guideline, for purification of nucleic acids from liquid biological samples, ports and channels for movement of liquids can range from about 1 millimeter to about 3 millimeters in diameter, such as from about 1.25 to 2 millimeters in diameter. Ports and channels for movement of gases can be somewhat smaller, for example on the order of 1 millimeter in diameter.

One or more surfaces of the body of the purification cartridge comprises holes that connect via conduits, channels, etc. to internal elements of the cartridge. These holes, or ports, provide access for fluids to the internal elements of the cartridge. In general, the cartridge comprises at least one inlet port for receiving a sample from an external source, at least one inlet port for receiving a fluid that is used in a purification scheme, and at least one exit port that allows waste material to exit the purification cartridge. In embodiments where a substance of interest is to be removed from the cartridge, the cartridge may include a substance collection port, which is accessible to the external environment. Although not required, inlet ports typically align and mate with exit ports from another element of the system, while exit ports align and mate with inlet ports of another element. For example, inlet ports for entrance of liquids used in automated purification schemes should align and mate with exit ports of a reagent pack, while waste exit ports of the cartridge should align and mate with inlet ports of the waste container. To better ensure proper movement of fluids within the system, the holes or ports on each element should align and mate with others in a fluid-tight seal. Thus, for example, the reagent pack, such as by way of flexible tubing, and the purification cartridge should mate and allow for transfer of buffers across mating surfaces without leaking or loss of buffer (and the resulting loss of pressure). Suitable seals, such as those made of plastic or rubber, may be included in the ports, if deemed advantageous. Where appropriate, the materials used for the connectors (e.g., male-female couplings) can provide the desired fluid-tight seal based solely on their intrinsic properties. In other situations, separate sealing elements (e.g., washers, O-rings) may be provided.

Within the body of the cartridge are one or more channels or conduits for transmitting fluids to and past various elements of the cartridge. For example, each port of the cartridge is connected to at least one channel, and the combination is used to introduce fluids into the cartridge or to remove fluids from the cartridge. In some instances, one or more channels lead to a filtration unit. Exiting each filtration unit is at least one channel, which may bifurcate to two or more separate channels, each of which may terminate at a different location. For example, where the filtration unit is a pre-filter, a single exit channel may bifurcate at a valve point, where one bifurcated channel leads to an exit port for waste removal and the other bifurcated channel leads to a second filtration unit. Likewise, a single exit channel from the second filtration unit may bifurcate at a valve point to an exit port for waste material and to a second exit port, which can be a substance collection port. It is to be noted that channels may split and join as necessary to achieve fluid routing needs according to the method implemented. Thus, multiple channels for transferring waste material may merge into a single waste channel that terminates at an exit port. As a general matter, the channels of the cartridge body are provided to move sample from its container through the cartridge and to move fluids from a reagent pack through the cartridge. The various permutations of channel size, shape, and contour are variable and can be selected by the practitioner based on the type of substance to be purified, the type of sample to be loaded, and other parameters.

Also within the body of the purification cartridge are one or more valves that are involved in regulation of fluid flow within the cartridge. In typical embodiments, multiple valves are disposed within the cartridge. The valves are actuated by computer controlled actuators and are opened and closed based on the programmed purification protocol. In preferred embodiments, the valves are rotary valves that rotate about a circle to open and close multiple conduits, each disposed at a different angle upon the circle. The rotary valves are accessible through a surface of the cartridge and can be rotated by external controlling means. To better ensure a fluid-tight seal at the valves, a valve cover or cap may be provided. The purification cartridge can comprise one or more motor and electronics units that mechanically drive the rotary valves. These motor units can comprise part of the cartridge, can be independent add-on units, or can comprise part of the instrument itself. In embodiments, they are independently removable from the cartridge and thus represent an optional element of the cartridge.

Conduits of the cartridge at times bifurcate and join. At various junctures of two conduits, such as at junctures where two different compositions meet and mix, mixing of the compositions can be accomplished by joining of the two (or more) conduits at various angles to effect mixing. In embodiments, the merging conduits can comprise a mixing apparatus that increases the amount of mixing of the compositions. For example, a structure representing a Tesla static mixer can be employed to cause mixing of two or more compositions from two or more conduits. Alternatively, for example, mixing can occur in a holding/mixing chamber that allows for receiving and mixing of fluid from two or more channels, or from the same channel at different times, and can hold an adequate volume to achieve mixing.

According to the exemplary embodiments, the means for receiving a blood sample may be any structure that allows for physical connection of a container containing blood to the purification cartridge. In some situations, the means for receiving a blood sample can be considered as an inlet port for the sample. Accordingly, it may be a well or other tubular structure into which a tube of blood (e.g., a Vacutainer) may be inserted. Although these elements may be provided independently, the means for receiving a blood sample may comprise, as an additional feature, means for heating the sample. Likewise, it may comprise a mechanical device to mix the sample and another composition to cause lysis of some cells (e.g., red blood cells) prior to passing the sample over a retaining means. In this way, there is less cellular material to be captured by the retaining means, and the overall efficiency of the system improves.

Typically, the means for receiving a blood sample comprises means for puncturing the container containing the blood such that the contained blood may exit the container and enter the purification cartridge. While any suitable object may be used to achieve this function, typically, the puncturing means will be a needle or other sharp object that can pierce a tube seal, such as a rubber (e.g., neoprene) stopper. The puncturing means may further comprise means for pressurizing the container. More specifically, containers containing blood drawn from patients typically are under a slight vacuum pressure. In order to cause the blood in the container to exit the container and move into the purification cartridge, it is often advantageous to create a positive pressure in the container, as compared to the purification cartridge, or at least provide a means for equalizing the pressure with ambient air pressure. The means for pressurizing the container may thus comprise a needle or other sharp object for puncturing the cap of a blood container, and a needle or other conduit that allows for pressure to be applied to the interior of the blood container. The needle or conduit should, of course, be connected to a source of pressurized fluid (preferably air or pure gas), or should allow for atmospheric gas to enter the blood container as needed. In essence, it is preferable to have “make-up” air enter the blood container as the blood exits the container.

According to the exemplary embodiments, the puncturing means of the means for receiving a blood sample is connected to the means for conducting all or part of the blood sample to a means for retaining at least some of the cells in the sample. Connection can be by any physical way that allows for a fluid-tight seal. For example, a needle may be connected by way of rubber tubing to a conduit etched into the purification cartridge, where the conduit leads from the tubing to a first reservoir, where a first step of purification occurs. As with all other connections in the system of the invention, connection of the puncturing means to the conducting means is by way of a fluid-tight seal.

In the exemplary embodiments, the purification cartridge thus comprises means for retaining at least some of the cells present in the blood sample. In general, the retaining means is a filter or series of filters (“filtration unit” or “pre-filter”) that entrap large materials, such as cells, based on size. The filtration unit comprises a solid support, as discussed above. In the exemplary embodiments, preferably, the filtration unit comprises filters having a size sufficient for entrapping white blood cells but permitting some or all of the red blood cells, lysed cell debris, and blood components to pass through. The filters thus act as a solid support for the cells. Within the context of a method of purification of RNA from blood cells according to the invention, the pre-filter provides an advantage over other devices and methods in the art in that it allows for removal of red blood cells and mRNA corresponding to globin genes, which can cause problems for analysis of white blood cell specific RNA. Numerous filters and combinations of filters can be used, with the goal being capture of cells that can subsequently be washed, if desired, and lysed on the filters to release the contents of the cells. As used herein with regard to certain embodiments, the capturing means is referred to as a means for capturing cells. In some embodiments, the means for capturing cells preferentially captures nucleated cells.

The pre-filter according to embodiments is a unitary element comprising one or more filter disks layered upon each other to form a multi-layered filtration unit. In certain embodiments, multiple (for example, 2, 3, 4, 5, or more) layers of a solid phase substrate, for example a filter, are used. For RNA isolation, cells (predominantly white blood cells) are typically retained on a first solid phase substrate, for example 47 mm diameter glass fiber filters (Whatman GF/D or Ahlstrom Paper Group 141). Cells are lysed on the first solid phase substrate, and the resulting cell lysate, including RNA, is released from the first solid phase substrate while DNA is retained. Associated with the pre-filter may be one or more screens, such as those made of plastic and having a pore size of 250 micrometers. The screen(s) can be included as physical support for the pre-filter and to assist in distribution of fluids over the pre-filter.

The cartridge can further comprise a binding means for the substance of interest. The binding means can be any element that binds the substance or binds non-target substances to provide purification of the substance. Where the substance of interest is a nucleic acid, and in particular RNA, the binding means can be a second filtration unit. In binding of RNA, glass fiber filters may be used. While not being bound to any particular mechanism of action, the second filtration unit is believed to function to bind RNA by adsorbing nucleic acids in a size-independent manner. It thus presumably works predominantly by chemical binding of substances, and not substantially by size exclusion. In embodiments, the second filtration unit comprises Whatman GF/F or Ahlstrom Paper Group 121 filters. It is to be understood that the second filtration unit may comprise any number of different solid phase supports, including, but not limited to, glass fiber filters, ion-exchange filters, and hydrophobic interaction resins, membranes, or filters. Likewise, any number of layers may be used (e.g., 1, 3, 5, 7, etc.).

Traditionally, filters are selected so as to have a pore size and composition that will act as an absolute barrier so as to prevent the material to be filtered (e.g., white blood cells) from passing through the filter material. For example, by selecting a filter material with a particular pore size it is possible to prevent materials with a particle size greater than the pore size from passing through or into the filter material. This concept is used in developing appropriate filters for the first filtration unit (i.e., the pre-filter or cell retention means) or the second filtration unit, if it is to be based on size-exclusion principles.

The retention or entrapment of the cells and nucleic acid by the filter may arise by virtue of a physical or size-related barrier relating to the dimensions of the filter material including the pore size and depth of the filter, or by other means. Without wishing to be bound by theory, it is thought that cells and large nucleic acid molecules may be physically associated with certain filters as well as chemically or otherwise tightly bound thereto. It is postulated that nucleic acid-nucleic acid interactions themselves are important in maintaining a sufficiently high cross-sectional area to retard movement of the nucleic acid through certain filters.

The pore size or particle retention rating of a first solid phase substrate intended for RNA purification from a sample is from 1.7 to 3 micrometers (um or microns), preferably from 1.9 to 3 um, and most preferably from 2.0 to 2.7 um. Preferably the pore size or particle retention rating of a second solid phase substrate for purification of RNA from a sample (and in particular for binding RNA from a sample) is from 0.5 to 1.8 um, preferably from 0.6 to 1.5 um, and most preferably from 0.7 to 1.6 um.

Filters useful for a first solid phase, that is filters useful for retaining a wide variety of cells types, including white-blood cells, include but are not limited to the Whatman GF/D glass fiber filter (a coarse porosity, a fast flow rate and a 2.7 um size particle retention value), Whatman QM-A (particle retention rating of 2.2 um), and Whatman EPM 2000 (particle retention rating of 2 um). Filters useful for a second solid phase (i.e., the second filtration unit), that is filters useful for retaining nucleic acids, include but are not limited to the Whatman GF/F glass fiber filter (a fine porosity, a medium flow rate and a 0.7 um size particle retention value), the Whatman GF/A filter (1.6 um size particle retention value), Whatman GF/B (1.0 um size particle retention value), Whatman GF/C (0.7 um size particle retention value) Whatman 934-AH (1.5 um size particle retention value), and Whatman GMF (1.2 um size particle retention value). Ahlstrom Paper Group also manufactures filters with similar properties to those offered by Whatman and can be used interchangeably with the Whatman filters in both filtration units.

Returning now to the means for capturing a cell, which can be envisioned in embodiments as a pre-filter, the means can include a multi-part filtration unit housed in a reservoir in the purification cartridge. Within this context, the cartridge body may comprise a conically-shaped reservoir, such as one that can be drilled or carved from the cartridge body block by a conically-shaped drill bit. The pre-filter can be designed to fit within this reservoir. Among the elements of the pre-filter are a conically-shaped fluid director, which can force sample and other fluids to flow to the perimeter of the reservoir by flowing down radial channels in the director. In contact with the fluid director is a proximal screen or mesh (e.g., a disk) that can filter out large particles and debris from the sample. The screen or mesh may be fabricated from any of a number of materials, including, but not limited to polypropylene and polyethylene. In use, the proximal screen is contacted by sample flowing down the conical face of the director, at the periphery or perimeter of the screen. The screen directs the flow of the sample across the solid substrate starting at the periphery and moving toward the center. Behind and in contact with the proximal screen is a filter or set of filters (i.e., solid support) that can filter, by size exclusion or other characteristics, substances in the sample. In embodiments, the filter(s) trap nucleated cells. A distal screen or mesh is behind and in contact with the filter(s), and serves as a support and a bridge between the filter(s) and the reservoir exit. In practice, sample or other fluid enters the reservoir by way of a central entrance hole at the apex of the conically shaped reservoir. Sample is channeled to the perimeter of the conically shaped reservoir substantially at the base of the cone. The configuration of the filtration unit causes sample to traverse the mesh/filter sandwich from the perimeter toward the center, trapping intact cells on the filter(s). Untrapped fluid and solid matter passes through the mesh/filter sandwich and exits the reservoir or chamber by way of an exit hole substantially at the center of the circle defining the distal portion of the chamber. The design of the filter unit allows for even distribution of sample over the filter, providing exceptional binding capacity and total yield of molecules of interest.

In preferred embodiments, the pre-filter is designed to filter 5 ml of blood, although smaller volumes (e.g., 3 ml or less) and larger volumes (e.g., 10 ml or more) can be accommodated by changing the number of filters or the surface area of the filters. Indeed, the size of the filtration unit may be altered infinitely to achieve purification of a desired volume of sample.

In yet another aspect of the invention, means for storing one or more liquid compositions is provided. In general, the storage means comprises one or more independent means for storing one or more liquid compositions, each of which comprises or is fluidly connected to at least one means for conducting the respective liquid compositions out of the storage means. In embodiments, the storage means can be considered to be a reagent pack. In embodiments, the means for storing liquids is a container that comprises an outer shell defining a shell for housing two or more inner compartments. As with other elements of the present system, the storage means can be fabricated from any suitable material or combination of materials, such as plastics, metals, and rubbers. In certain embodiments, the storage means is a container made of hard, resilient plastic that can not only house internal compartments but can provide a level of protection to those compartments as well.

In some embodiments, the storage means is a unitary article of manufacture that comprises an outer shell and one or more inner dividers. The number of dividers present, and the size of the internal compartments can be varied and selected by the practitioner based on the type of purification scheme envisioned and the amount of the various fluids needed to achieve the purification. Thus, the number of internal compartments may vary from as few as one to ten or more. The inner compartments comprise independent sub-containers for various fluids, including, but not limited to, buffers, lysis solutions, organic solvents, water for purification of target substances, and waste fluid. The sub-containers may be defined by the exterior and interior walls of the container, or may be defined by other walls provided at least in part by additional elements. In certain embodiments, a cylinder or syringe-like sub-container is provided for each fluid to be contained, where each cylinder can be independently regulated for pressure and delivery of the fluid contained in it, such as, for example, by actuation of a plunger or piston by pressure supplied by a pump. For example, in embodiments, a gas or air in general is used to pressurize the sub-containers and force fluid from the sub-container. In other embodiments, the container is a collapsible bag that can change volume in response to removal of fluid from it. In yet other non-limiting embodiments, the sub-container is a rigid-walled container that can withstand pressure changes when fluid is removed. Yet again, the sub-container may have a vent to receive make-up air as a fluid is removed.

The storage means can also comprise one or more conduits for delivery of fluids to the exterior of the storage means. For example, the storage means can be a container comprising two or more compartments, each containing a different fluid for use in a purification scheme. Tubing can be connected by way of a fluid-tight seal to an exit port for each compartment, and the tubing can provide an exit port for movement of fluids out of the container. The exit port may comprise means for creating a fluid-tight (e.g., water-tight) seal with a mating surface, such as an entrance port for a purification cartridge. In some embodiments, the tubing is configured on a reagent pack to align with the head of a peristaltic pump to deliver one or more fluids from the reagent pack to a purification cartridge. In embodiments, the termini of all tubing are aligned along a plane to allow for mating with a purification cartridge.

In embodiments, the storage means further comprises means for replacing volumes of liquid removed from the storage means to maintain a suitable pressure in the storage means. The storage means includes means for allowing fluid to exit the storage means. In embodiments, the storing means comprises a reagent pack comprising one or more containers that contain liquid compositions, each of which are connected to a tube, such as a piece of flexible, compressible tubing, that acts as a conduit from the container to one or more exit ports on the reagent pack. In some embodiments, the reagent pack comprises one or more containers that receive and contain waste products from a purification process.

The storage means can comprise, on an outer surface, one or more means for attaching it to an instrument of the invention, a purification cartridge of the invention, or both. As with other attachment means discussed herein, this attachment means can be fabricated from any suitable material in any suitable form. Preferably, the attachment means is fabricated to mate or align with a complementary structure on the instrument or cartridge. In some embodiments, the mating surface of the reagent pack is designed to include a portion that aligns with and interacts with at least a portion of a pump at the surface of the instrument. More specifically, one configuration of the reagent pack includes placement of flexible, compressible tubing along a surface of the pack. The tubing will be exposed to the exterior in such a manner that, when coupled to an instrument with at least a portion of a pump exposed on a surface, the tubing of the reagent pack can contact the pump in a manner that allows the pump, when running, to force fluid through the tubing from the sub-containers to the exterior of the reagent pack, and preferably into a purification cartridge. The number of flexible tubing/pump head connections are not limited in theory, although the size of the pumps might be a limiting factor in accommodation of all within the instrument housing. The size of the tubing is not critical, although in some situations it can be advantageous to use a relatively small inner diameter to reduce “dead volume” and inefficiencies in the method.

In addition to configurations that present a surface having tubing exposed for interaction with a pumping mechanism, in embodiments the reagent pack comprises one or more ports that align with one or more ports on a purification cartridge. Preferably in these embodiments, each sub-container of the reagent pack is provided with its own exit port, which aligns and physically contacts one entrance port of a purification cartridge. As with all other connections discussed herein, it is preferred that the connection between the exit port of the reagent pack and the entrance port of the purification cartridge be a fluid-tight seal, such as by use of a male-female connection and/or by use of compressible seals (e.g., O-rings or washers).

The reagent pack can be designed to be removably attached to the instrument, the purification cartridge, or both. It further may be designed to be disposable, having a useful life of anywhere from one purification run to ten purification runs or more. In general, the number of purification runs is not critical to the reagent pack; however, from a practical standpoint, the amount of volume held by the reagent pack when fresh will typically be the limiting factor, as the reagent pack will find use in the context of a portable consumable. While there is no upper or lower limit to the amount of volume the reagent pack may contain, it will typically contain on the order of 40 liters or less of liquid, such as 20 liters or 10 liters. Of course, where desired, the system of the invention can be designed as a larger system for processing multiple samples using the same purification scheme. Thus, in embodiments, the reagent pack may comprise significantly more volume than 10 liters, for example 20 liters, 40 liters, 50 liters, or more.

In a further aspect, the invention provides means for receiving waste products from the purification means, the storage means, or both. In general, the waste receiving means comprises at least one container that receives and stores waste materials from the purification means, the storage means, or both. In some embodiments, the waste receiving means is a compartment disposed within the storage means (e.g., the reagent pack).

Typically, the waste receiving means is a container that comprises at least one inlet port disposed on an outer surface of the means. The inlet port is fluidly connected to at least one container by way of tubing or other suitable conduit. In use, the waste container accepts waste products from a purification scheme performed on a purification cartridge, most or all of which ultimately derive from a reagent pack connected to the purification cartridge. In some embodiments, the container comprises an exit port, such as a vent, that allows a connection to the external environment, which can assist in maintaining suitable pressure in the container. In other embodiments, the container is a deflated flexible bag that can expand as fluid is introduced into it.

The waste receiving means can be connected to a pressure generating means, which produces a negative pressure within the container. Alternatively, the waste receiving means may be fabricated to contain a vacuum. In either embodiment, the vacuum pressure is made available to one or more conduits of the purification cartridge upon connection of the purification cartridge to the waste receiving means. This negative pressure may act to “pull” fluids through the purification cartridge. In exemplary embodiments, the negative pressure generating means comprises a peristaltic pump.

In certain configurations, the waste containment means comprises multiple containers. In these embodiments, certain waste materials can be segregated and separately contained. For example, where a purification protocol calls for use of a toxic or otherwise hazardous substance, that substance can be contained in a separate container from other, non-hazardous substances. Such a segregation can be helpful in complying with certain local, state, or federal requirements.

In an additional aspect, the invention provides means for controlling a process of purification of a substance from a sample. In general, the means for controlling a purification process comprises computer software (e.g., a program) that executes on a computing device to effect one or more steps in a purification process. The means for controlling typically comprises software that, when executed by a computing device, results in control of one or more mechanical devices of the system.

According to this aspect of the invention, any suitable computing device running any appropriate software may be used. The type of computing device and software, including the type of operating system, computer language in which the software is written, and type of hardware employed is not critical. Those of skill in the art may select from among many combinations of hardware and software available in the art to achieve a suitable computing device.

In view of the adaptability of the present system for purification of any number of target substances, various computer programs will be required. It is to be noted that, unless a particular unexpected problem is encountered, none of the programs require unusual coding skills or excessive lengths of time to develop. Rather, upon determination of a suitable purification protocol, it is a matter of ordinary skill in the art to develop a computer program to implement the protocol. For example, it is a simple matter to develop a computer module that can control the timing and movement of one or more valves of the system, control the pumping action of one or more pumps that move fluids from the storage means to the purification means, etc. It is thus unnecessary to disclose particular computer code to allow one of skill in the art to develop a computer program according to the present invention.

In another aspect, the invention provides an automated method of purifying or isolating one or more substances from a sample. While not so limited, typically, the method is a method of purifying or isolating a substance from a sample comprising one or more biological molecules, such as a nucleic acid or protein. In general, the method comprises: exposing a sample comprising one or more substance of interest to a filtering means such that the substance is captured by the filtering means; releasing the substance of interest from the filtering means; and exposing the substance of interest to a binding means. In embodiments, the substance of interest is a biological molecule found in a cell. In these embodiments, the step of exposing the sample to the filtering means results in binding of the cell to the filtering means, and the method further comprises lysing the cell to release the substance of interest. In the method, all of the steps are performed automatically by a machine, such as one controlled by a computer program. In other words, none of the steps of the method requires human interaction or human action, although certain optional steps (e.g., providing a sample) may include some human action.

In various embodiments, the present invention provides automated methods for separating, purifying, and/or isolating biological molecules and/or cells from a sample. Accordingly, in one aspect, the invention provides a method of isolating biological molecules, such as nucleic acids, proteins, and blood components from a sample. In general, the method comprises providing or obtaining a sample comprising at least one biological molecule or cell and purifying at least one biological molecule or cell from the sample. The method may also encompass inserting a sample comprising at least one biological molecule or cell into a system that will automatically isolate at least one biological molecule or cell from the sample. The sample is usually at least 1 milliliter (ml) in volume. For example, milliliter quantities of whole blood comprising leukocytes or cultured cells can be used as the sample in the method to isolate nucleic acids.

The methods of the apparatus are automated, meaning that the steps of the methods occur mechanically and substantially without the intervention of a human. In a preferred embodiment, the methods of isolation take place in the system of the present invention. In this case, the method comprises adding the sample to the instrument and allowing isolation of at least one biological compound or cell to occur. As such, “automated” includes a meaning by which, in general, no human intervention is required after inserting the sample into the machine until the purification of at least one biological molecule or cell is complete. In the system of the invention, the sample and/or biological molecules of interest are primarily transferred through the instrument by the mechanical displacement of liquids.

In one embodiment, the method of the present invention comprises adhering or binding of at least one biological molecule to at least one solid substrate. Preferably, the binding occurs in the presence of salts and an organic solvent. For example, the method comprises exposing a sample, preferably at least 1 ml in volume, comprising nucleic acids to a solid substrate in the presence of an appropriate mixture of salts and organic solvent such that some or all of the nucleic acids bind to the solid substrate. As described in detail below, this method can be adjusted to selectively bind predominantly single-stranded nucleic acids or double-stranded nucleic acids.

In another embodiment, the method comprises a way of isolating a biological molecule or cell using a prefilter. The method comprises contacting at least one biological molecule or cell to at least one prefilter. This method can employ the prefilter to separate large biological molecules and/or can use the prefilter as a binding support during lysis of selective biological molecules. For example, cells found in blood, such as red blood cells and white blood cells, can adhere to the prefilter while smaller blood components flow through the prefilter. Therefore, this embodiment may be used to selectively separate red and white blood cells from the rest of the whole blood components. The cells that bind can be removed from the prefilter and retained for further use or can be selectively lysed by adding different lysis buffers to the prefilter for isolation of nucleic acids. For example, this embodiment may be used to purify white blood cells from whole blood by retention of red and white blood cells on the prefilter and subsequent lysis of red blood cells. The white blood cells can then be removed from the prefilter, resulting in a composition that is primarily white blood cells. As another example, the prefilter may be used in a method to separate bigger biological molecules, such as genomic DNA, from smaller molecules, such as RNA. Larger biological molecules will not be able to flow through the prefilter while smaller molecules will be able to pass through. In this way, the larger biological molecules may be selectively isolated and/or the smaller biological molecules may be purified from the larger ones.

Another method of the invention comprises an automated system that uses both a prefilter and a solid support substrate to isolate a biological compound. In this case, a sample, preferably at least 1 ml in volume, is inserted into the system, and at least part of the sample goes through both a prefilter and a solid support substrate. As the sample contacts the prefilter, some biological compounds are retained by the prefilter while others flow through. For example, when the sample is whole blood, blood cells will be retained by the prefilter. Red blood cells can be lysed and remnants of the red blood cells can be washed off the prefilter. Subsequently, nucleated white blood cells can be lysed and the released nucleic acids are either caught by the prefilter or dispersed into the lysate. If only white blood cells are present in the sample, the step in which red blood cells are lysed can be eliminated. Large nucleic acids that are caught by the filter can then be purified from the rest of the biological molecules. The lysate that flows through the prefilter can be contacted with a solid support substrate in the presence of salts and organic solvent to allow selective binding of some other biological compounds, which can then be eluted off the substrate in a final purification step. This method allows isolation of biological molecules and/or cells within an automated system, wherein the purification steps are fully mechanized after insertion of the sample. After isolation, the biological molecules and/or cells of interest can be removed from the instrument.

In one exemplary embodiment, the method of the invention is used to purify nucleic acids. The method comprises isolating or purifying at least one nucleic acid from a sample. In an optional embodiment, the sample is obtained, provided, or in some way procured prior to being purified. In this embodiment, the nucleic acids in a sample bind, adhere, or are caught in a prefilter. The nucleic acids may be intracellular, meaning within a cell, or extracellular, meaning outside of a cell. If the nucleic acids are found inside a cell, the cell can be lysed while still on the prefilter using a lysis buffer. When the sample is blood, red blood cells can be lysed prior to breakage of white blood cells so that contaminants found in red blood cells such as heme from hemoglobin and RNases can be removed. Subsequent lysis of white blood cells releases nucleic acids onto the prefilter and/or into the solution. Larger nucleic acids, such as genomic DNA, are caught by the prefilter and do not flow through. Smaller nucleic acids, such as RNA, will not be bound or caught by the prefilter and can pass through. Nucleic acids that do not go through the prefilter can be retrieved at this point as a way of purifying the larger nucleic acids. Therefore, this embodiment is a way of isolating larger DNA molecules from the sample. For isolation of smaller nucleic acids, such as RNA, for example, the lysate can be contacted with a solid substrate in the presence of an organic solvent and salts. Under certain concentrations of salts and organic solvent, RNA will bind to the substrate. After optional washes, the RNA can be eluted in a buffer and therefore, in one embodiment, the method comprises purification of RNA molecules from a biological sample. The isolated RNA is pure enough to be used directly in assays or used in further isolation steps, such as to purify mRNA from the total RNA.

In a preferred embodiment, the method is a method for purifying RNA from white blood cells from a sample comprising whole blood. The method is an automated method that includes: mixing of the whole blood with a red blood cell (RBC) lysis buffer and exposing the mixture to a filter, which traps unlysed cells, including both red blood cells and white blood cells; exposing the filter/cell complex to additional RBC lysis buffer either as a continuous stream of buffer flowing over the filter or as a series of two or more batches of RBC lysis buffer that are permitted to remain in contact with the filter for a period of time to effect lysis of RBC; washing the filter one or more times with phosphate buffered saline (PBS) or a functionally equivalent aqueous solution or water; drying (at least partially) the filter by passing air or another gas over the filter; lysing white blood cells (WBC) bound to the filter by exposing the filter to WBC lysis buffer as a continuous flow of buffer or as a series of two or more batches of WBC lysis buffer that are permitted to remain in contact with the filter for a period of time to effect WBC lysis; washing of the filter/lysed cells complex with water one or more times to release entrapped RNA from the filter; mixing the WBC lysate with sulfolane to create a mixture; exposing the mixture to a second filter, which binds RNA present in the mixture; washing the filter/RNA complexes with a low salt wash solution at least one time, with optional air drying of the filter after each wash; exposing the filter/RNA complexes to ethanol, with optional air drying of the filter after exposure to ethanol; exposing the filter/RNA complexes to water to release RNA bound to the filter; and collecting the released RNA.

In the embodiment described immediately above, washing of the first filter can be accomplished with PBS in a series of washes with equal volumes of PBS, such as 8 cycles of washing, each with 1.5 ml of PBS. It has been found that such successive small-volume washes provides superior clearing of RBC debris from the filter than a single, continuous washing by way of flowing of PBS across the filter in a stream, even where the volume of PBS continuously flowing across the filter is twice the volume of the total volume of the successive small-volume washes. It has also been found that air drying of the filter between each washing batch improves total yield of RNA and generally improves the performance of the automated method. In this regard, it has been found that the pressure used for flowing the air/gas over the filter can be increased, or ramped-up, on successive air purges. While not being limited to any particular mode of action, it is envisioned that each successive washing removes more debris or other unwanted material from the filter, allowing for more air to flow across the filter at a given pressure level. One particular advantage to ramping up of the air purge pressure is the ability to effectively dry the filter in a shorter period of time, which can be important when isolating RNA, as the RNA profile of a given cell or group of cells can change while the unlysed WBC are entrapped on the filter. Preferably, WBC are lysed within ten minutes of removal from a patient; minimizing air drying times, in conjunction with other features of the automated method and system of the invention, allows for such rapid lysis.

Furthermore, it has been found that a series of washes of the filter with water after lysis of WBC provides superior yields of RNA, as compared to yields without the washes. More specifically, it has been found that lysis of WBC entrapped on the filter in a series of washes with water results in elution of predominantly RNA, while DNA entrapped by the filter remains substantially bound to the filter. The RNA eluted from the membrane is of substantially the same high quality as the RNA released during lysis of the WBC. An increase in yield of high quality RNA on the order of 20%-30% has been seen using three washings of the filter with water. Where washing with water is performed, the eluate should be mixed with WBC lysis buffer and combined with the original eluate for further processing.

According to preferred embodiments of the invention, RNA released from WBC is bound to a second filter to allow for improved purification of the RNA. Bound RNA is washed to remove unwanted substances that are present on the filter and in the liquid composition in which the RNA was initially found. The washed RNA is then exposed to ethanol (preferably 95% or greater in concentration, such as 100% ethanol) to dry and stabilize it. It has been found that RNA bound to the filter may be immediately removed from the filter after removal of the ethanol. That is, it has been surprisingly found that the filter/RNA complexes do not need to be washed with an aqueous solution, such as a low salt wash buffer, to remove ethanol and re-hydrate the RNA prior to elution from the filter. Rather, RNA may be eluted from the filters after simple removal of the ethanol from the filter/RNA. According to the automated method of the invention, the ethanol can be removed by purging of the filter chamber with air. This results not only in removal of the ethanol, but drying of the filter as well. Results of studies have shown that there is no significant difference in the quantity or quality of RNA eluted from the filter when processed with or without washing of the filter with low salt buffer prior to elution of the RNA.

The sample volume used for the method of the present invention is generally more than or equal to about 1 ml, such as from about 1 ml to about 10 ml and from about 10 ml to about 15 ml. However, as stated above, volumes can be varied in conjunction with the sizes/diameters/volumes of solid supports, etc. In a preferred embodiment, the sample volume is about 5 to about 10 ml, such as the volume that fits into a standard Vacutainer tube. With the use of larger sample sizes, one can isolate a greater number of biological compounds or cells than if one used microliter quantities of a sample, such as those added to microtiter plates. Of course, some embodiments of the method can be envisioned to utilize even greater volumes of the sample, such as if the prefilter and mineral substrate are larger than shown herein. The methods of the present invention may be used in large scale purifications of biological compounds. For example, if the system is made to handle liter quantities of sample, the methods may be modified for the bigger instrument.

In one embodiment, the method of the present invention can be used to purify nucleic acids. In a preferred method, single-stranded RNA is separated from double-stranded nucleic acid, preferably DNA. If DNA is present in a single-stranded form, it may be separated from double-stranded DNA, as well as from double-stranded RNA. RNA that can be isolated by this method includes mRNA, tRNA, rRNA and noncoding RNA such as snRNA, snoRNA, miRNA, and siRNA. The size of RNA that can be isolated by this method is not particularly limited, but typically ranges from about 20 nucleotides (such as some siRNA) to more than about 5 kb or 6 kb (such as some mRNA). It is envisioned that if total RNA is isolated, subsequent or concurrent steps can be added to the method to allow purification of mRNA. For example, oligo (dT) molecules, such as biotin labeled oligo (dT) nucleotides and streptavidin coated magnetic beads, can be used to isolate mRNA molecules. In addition, subsequent steps that add more organic solvent or a different organic solvent may selectively allow binding of different kinds or sizes of RNA molecules to a mineral substrate. Generally, steps to the methods can be added that further purify or isolate specific biological molecules and components of the system can be added or modified to incorporate the changes in the methods.

One embodiment of the invention provides an automated method of isolating RNA from a cell sample comprising the following steps. A sample is applied to a first solid phase substrate such that the cells of said cell sample adhere to said substrate. The cells are lysed on the first solid phase substrate to form a cell lysate comprising RNA. The cell lysate is passed through the substrate and is collected. The cell lysate is applied to a second solid phase substrate to immobilize the RNA. The RNA is eluted from the second solid phase substrate. According to the method, all steps are performed without human intervention. In some embodiments, the steps of applying and/or collecting can involve human actions.

In one embodiment, the method further comprises the step of selectively lysing red blood cells prior to applying the sample to a first solid phase substrate, or lysing red blood cells captured on the first solid phase substrate, or a combination of both. In another embodiment, the cells, such as white blood cells or other cells containing a nucleic acid of interest, are lysed by the addition of a lysis solution to form a cell lysate. In another embodiment, sulfolane is added to the cell lysate prior to the step of applying the cell lysate to the second solid phase substrate.

The method of the invention may further comprise the step of isolating DNA from the first solid phase substrate following the step of lysing the cells on the first solid phase support to form a cell lysate comprising RNA. In addition or alternatively, the method of the invention may further comprise any of the following steps: isolating protein from the cell sample; washing the cells following the step of applying the sample to a first solid phase substrate and before the step of lysing the cells on the first solid phase substrate to form a cell lysate comprising RNA; washing RNA immobilized on a second solid phase support to remove contaminants; eluting the RNA from the second solid phase support; and optionally eluting DNA from the first solid phase support.

The invention also provides an automated method of isolating RNA from a cell sample comprising the following steps. A sample is applied to a container comprising a first and second solid phase. The container is connected to an instrument that provides the force by which the cell sample and solutions are moved through the container, and the cells are adhered to the first solid phase substrate. The cells are lysed on the first solid phase substrate to form a cell lysate comprising RNA, while DNA is bound to the first solid phase support. The cell lysate is passed through the first solid phase substrate and is collected. The cell lysate is mixed with appropriate materials (e.g., sulfolane) allowing application to a second solid phase substrate to immobilize the RNA. The immobilized RNA is eluted from the second filter and DNA is eluted from the first solid phase support. In another embodiment, the automated method further comprises the step of isolating protein from the cell sample.

In certain embodiments, the isolation methods include a step of exposing the cell sample to at least one solid phase substrate. In other embodiments, the isolation methods include a step of exposing the cell sample to more than one solid phase substrate.

In some embodiments, the invention relates to methods of isolating material from a sample. In particular, the invention relates to methods of isolating nucleic acids from a sample.

The invention further relates to methods of isolating RNA from a sample, such as one comprising blood or a blood cell. For isolation of white blood cell material from whole blood, EDTA or heparin anti-coagulated whole blood can be added to a hypotonic solution, for example (0.15 M ammonium chloride, 1 mM potassium bicarbonate, 0.1 mM EDTA, pH 7.3) and incubated until the turbid whole blood clears due to red blood cell lysis.

To perform the methods of the invention within the context of the system for purification of a nucleic acid from a cell, the cell sample is introduced into the purification cartridge. The cartridge is connected (either before or after applying the sample) to an instrument that forces liquids to flow from a reagent pack to the cartridge. The instrument forces air, and not liquid, through a reagent pack to deliver liquid reagents and effect purification, for example, through macro-channels. Fluid does not enter the instrument, thereby eliminating the need to clean internal tubes and valves, and preventing cross-sample contamination.

In another embodiment, fluids, including liquids and gases, are pumped through the system through the use of a pump, such as a peristaltic pump. Typically, the liquids are pumped from one or more reagent packs through the cartridge and into a waste pack. The gases are pumped either from a contained source of the gases or from ambient air, which is preferably filtered to remove substances that could interfere with purification of the substance of interest or degrade the quality of the substance of interest.

In one embodiment, the automated isolation method takes no longer than 3 hours, for example, 3 hours, 2 hours, 1 hour or less. For example, the automated isolation methods can take less than an hour, for example 59, 58, 57, 56, 55, 50, 45, 40, 35, 30, 25, 20, 15, 10, 9, 8, 7, 6, 5, 4, 3, or 2 minutes, or 1 minute or less.

According to embodiments of the method of the invention, a cell sample is passed through a first solid phase substrate wherein cells are retained. In some embodiments, the cell sample is a blood sample that comprises red blood cells. In such situations, the sample can be mixed with a red blood cell lysis solution, allowing lysis of red blood cells. White blood cells (WBC) remain intact and are captured on the first solid phase substrate. The WBC retained on the first solid phase substrate are washed in an appropriate buffer, for example PBS. The WBC are lysed by the addition of an appropriate lysis buffer (for example, 4 M guanidine thiocyanate, 0.05% sarkosyl, 0.01% Antifoam A, 0.7% beta-mercaptoethanol, 1% Triton X-100). The resulting cell lysate contains RNA and passes through the first solid phase substrate. DNA is retained by the first solid support substrate.

RNA in the flow-through from the first solid phase substrate lysate is isolated by adding sulfolane to the cell lysate and passing the resulting mixture over a second solid phase substrate. The RNA is retained by the second solid phase substrate and can be washed in an appropriate buffer, for example 2 mM Tris, pH 6 to 6.5, 20 mM NaCl, 50-60% ethanol). The RNA is eluted from the second solid phase substrate by the addition of the appropriate solution, for example RNAse-free water.

In certain embodiments of the invention, DNA is isolated from the first solid support substrate by elution with heated or room temperature water or low ionic strength buffer, such as 10 mM Tris, pH 8. The DNA may be isolated from the solid support by movement of liquid in the same direction or opposite direction of fluid flow during DNA binding. According to this method, DNA yield is directly proportional to the time and temperature that the low ionic strength buffer or water is in contact with the second solid phase substrate. Alternatively, DNA attached to glass fiber can be dislodged by sonication of the filter followed by elution with heated or room temperature water or low ionic strength buffer. DNA yield is typically proportional (e.g., directly proportional) to sonication time and intensity and temperature and time of water or buffer heating.

The size of the genomic DNA can be reduced for efficient elution from glass fiber, for example by using a rare cutting restriction enzyme(s) and/or DNase at optimal concentrations and incubation times. Following enzyme treatment, the sample may be heated to inactivate the enzyme(s) and water or low ionic strength buffer is used to flush the DNA fragments from the filter. Also, depurination of DNA at pH<3 followed by strand scission at pH>12 will also lead to production of small DNA fragments that can be eluted from the glass fiber filter.

As mentioned above, the invention provides for automated methods of purifying material from a sample. In one embodiment, any one of RNA, DNA, protein, or another biomolecule of interest are isolated by the automated methods of the invention. In one embodiment, any combination of RNA, DNA, protein, and another biomolecule of interest is isolated, either sequentially or simultaneously, using the automated methods of the invention. Typically, where protein is to be isolated, it does not bind to the second solid support, but rather is found in the flow-through fraction.

The invention thus provides a method of isolating material from a cell or cell lysate. In a preferred embodiment, the invention provides a method for isolating RNA from a cell or a cell lysate. It is to be noted that, according to the method of the present invention, all of the steps of the method are performed without the need for centrifugation and/or human interaction.

A preferred embodiment of the method of the present invention will now be described in detail in which nucleic acid is isolated from whole blood or cultured cells comprising blood cells using a fully automated process. The method comprises insertion of a sample containing whole blood or cultured cells into the system, capture of blood cells on a prefilter, optional washing of the prefilter, optional addition of a red blood cell lysis buffer to the same prefilter, addition of a white blood cell lysis buffer to the same prefilter, collection of lysate, addition of an organic solvent to the lysate, binding of nucleic acid to a solid support, such as a mineral substrate, optional washing of the mineral substrate, and elution of the nucleic acid. In the method of isolation of larger DNA, such as genomic DNA, the method comprises insertion of a sample containing whole blood or cultured cells into the system, capture of blood cells on a prefilter, optional washing of the prefilter, optional addition of a red blood cell lysis buffer to the same prefilter, addition of a white blood cell lysis buffer to the same prefilter, and elution of DNA from the prefilter by chemical and/or physical means. Although nucleic acid isolation from whole blood or cultured cells will be described, many of the parameters discussed apply to other embodiments of the methods of the present invention.

In the first step of this specific method, a sample containing whole blood or cultured cells is added to the instrument of the present invention. The sample may be in a tube, such as a Vacutainer tube, or in any other container that fits into the system. By “added”, it is meant that the container or containers comprising the sample can be manually inserted into the instrument or that an automated insertion can occur using a biorobot or other automated method. In other words, the method of the present invention may be used by itself or may be part of a larger automation method. For example, whole blood may be added to tubes via automation and then the tubes containing the whole blood may be automatically inserted into the instrument. In addition, after isolation of the biological molecules or cells, samples may be removed from the instrument using automation.

In the next step of this method, the sample is transferred by the machine to a chamber containing a prefilter. In general, the step of prefiltration comprises contacting the sample comprising at least one biological molecule or cell with at least one prefiltration substrate for a sufficient amount of time and under appropriate conditions to allow for capture of at least one biological molecule or cell in the sample by the prefilter. The step also comprises separating the unbound sample from the prefiltration substrate and bound biological molecule(s) or cell(s). Separation of the remaining sample from the prefiltration substrate comprising at least one biological molecule or cell can occur using any suitable technique, including, but not limited to, gravity, centrifugation, positive air pressure, and/or vacuum etc. In a preferred embodiment, the system uses pressure to force liquid through the pre-filter, both during application of the sample and during subsequent separation (e.g., by way of washing of the filter). A given volume of liquid, and in particular embodiments a washing solution, may be applied to or forced through the pre-filter as a single unit of liquid or as multiple sub-volumes of liquid, with pauses between application of each sub-volume of liquid. For example, a wash solution may be applied to cells/sample bound to the pre-filter, optionally allowed to remain in contact with the pre-filter for a pre-set amount of time, then removed from the pre-filter. The filter may then optionally be purged of wash solution by passing air or another gas over the filter. Then, one or more subsequent washes with wash solution may be performed. Subsequent drying of the pre-filter with air or another gas can then be performed.

Specifically for the isolation of nucleic acids from whole blood or cultured cells, larger blood components, such as red blood cells, white blood cells, and platelets, are retained by the prefilter and the remaining components, such as blood plasma, serum, and media from the cultured cells, pass through the prefilter in an automated fashion. Among other things, this step partially purifies the nucleated white blood cells and allows at least 1×10⁷ white blood cells to be processed on a single subsequent mineral support. The substrate utilized for the prefilter step can be any material that retains larger particles, such as blood cells and genomic double-stranded nucleic acid, but allows smaller biological molecules to pass through the prefiltration substrate. The substrate for prefiltration preferably may include a single or a combination of materials such as porous polyethylene frits, glass fiber, and cellulose acetate. The prefiltration substrates can be provided in any shape or size. For example, they can be provided in a combination of filter and an insert (collar) or polyethylene frit, which can be used for retaining the filter and, in embodiments, providing a filtering function. Of course, any number of configurations and combinations can be used for the prefilter as long as blood cells are retained and smaller blood components pass through. In some embodiments, the prefilter comprises one or more (e.g., two, three, four, etc.) glass filters, such as Whatman GF/D, or Ahlstrom Paper Group (Mount Holly, Pa.) 141 filters.

As discussed above briefly, after retention of the blood cells on the prefilter, the prefilter may be optionally washed by mechanical means to reduce the contaminants on the filter. Any washing solution that allows the blood cells to be retained on the filter can be used. For example, phosphate buffered saline may be used.

After the capture of blood cells onto the prefilter and an optional wash step, red blood lysis buffer can be added in an automated fashion to allow disintegration of the red blood cells on the prefilter. In embodiments, most or all of the red blood cells have been lysed prior to reaching the pre-filter by mixing of the sample with red blood cell lysis solution, and their contents passed through the pre-filter without binding. Any lysis buffer that will allow lysis of red blood cells but allow white blood cells to remain intact can be used in this step. For example, a specific lysis buffer that can be added is comprised of 0.15 M ammonium chloride, 0.001 M potassium bicarbonate, and 0.0001 M EDTA, pH 7.2-7.4. As an optional wash step, the prefilter can be washed, for example with more red blood lysis buffer, to further reduce contaminants. Of course, if red blood cells are not present in the initial sample, the addition of red blood lysis buffer may be eliminated in the method.

Once lysis of red blood cells has occurred and optional washing performed, white blood cell lysis solution can be mechanically added to the same prefilter to release nucleic acids from the white blood cells. For example, 4 M guanidine thiocyanate, 1% Triton X-100, 0.05% sarkosyl, 0.01% Antifoam A, and 0.7% beta-mercaptoethanol can be added as a white blood cell lysis solution. Large, predominantly double-stranded DNA, such as genomic DNA, that is released from the white blood cells is retained on the prefilter, while smaller nucleic acids, such as RNA and small DNA molecules, pass through the prefilter. Optional washing of the pre-filter one or more times with lysis solution, wash solution, or water, for example, can improve the total yield of RNA from the pre-filter. Where optional washing is performed, drying of the pre-filter with air or another gas may be performed between two or more of the washes.

Thus, in the method, the molecules captured by the prefilter can be released at a desired time by chemical and/or physical means. One simple and gentle way to remove the captured material is to flow a liquid across the prefilter in the opposite direction from the original filtration in the instrument. Doing so will dislodge a substantial portion of the entrapped material, which is then substantially purified from smaller material (for example, DNA is now purified from contaminating RNA). The macromolecules separated from the prefilter then can be directly analyzed or can be further purified by a variety of methods, including but not limited to being adsorbed to a mineral substrate in the presence of an appropriate mixture of organic solvent and an optimal concentration of salt or salts.

Depending on the sample constitution, after prefiltration, the flow-through fraction may comprise predominantly smaller double-stranded nucleic acids, such as small DNA molecules and/or RNA molecules. In general, the small DNA molecules that are found in the flow-through fraction are about 6 kb or less, such as from about 6 kb to about 4 kb, or from about 4 kb to about 1 nucleotide. In preferred embodiments, the DNA molecules are about 1 kb or less. The size of RNA that is in the flow-through fraction and therefore can be isolated by this method is not particularly limited, but typically ranges from about 20 nucleotides (such as some siRNA) to more than about 5 kb or 6 kb (such as some mRNA).

In some embodiments, the methods of the invention comprise exposing the flow-through fraction (eluate) to a second substance that binds biological molecules, such as a solid support or substrate that binds nucleic acids. For example, in a preferred embodiment, the invention provides a method of isolating or purifying nucleic acids, including single-stranded and double-stranded nucleic acids, using chaotropic salts and organic solvent. The method comprises exposing a sample comprising the nucleic acids to be isolated or purified to at least one solid substrate (also referred to herein as a mineral support or solid support), wherein the exposing conditions comprise an appropriate mixture of salts, especially chaotropic substances, and organic solvent, such that the nucleic acids are adsorbed on the substrate. Preferably, the mixture is an aqueous mixture. Optionally, the adsorbed sample on the substrate is washed with buffer or another aqueous composition after adsorption. In addition, in methods for isolating or purifying RNA, DNA molecules that are also adsorbed to the substrate can be removed by exposing the support and bound material to DNase (preferably RNase-free) under suitable conditions and for an adequate amount of time for digestion of the DNA to occur. Conversely, in methods for isolating or purifying DNA, RNA molecules that are also adsorbed to the substrate can be removed by exposing the support and bound material to RNase (preferably DNase-free) under suitable conditions and for an adequate amount of time for digestion of the RNA to occur.

In a preferred embodiment, the biological molecule being isolated is RNA. When the RNA and the solid support substrate, which is preferably silica-based such as glass filters, are exposed to each other in the presence of a chaotropic or other useful salt as previously described and an adequate amount of organic solvent, the majority of the RNA becomes bound to the substrate. In this context, the term “majority” means that more than 50% of the RNA molecules are bound to the mineral substrate, such as in some cases, more than 80% and in other cases, more than 90% and approaching 100%. Those of skill in the art can immediately recognize all of the particular values encompassed by this range, and thus each particular value need not be specifically recited herein.

The method may comprise combining the sample eluted from the prefiltration solid support substrate with organic solvent before exposing the resulting sample to the second solid support substrate under conditions wherein the biological molecule of interest binds to the second solid support substrate. In these embodiments, the organic solvent is typically added by mechanical means after prefiltration of the sample. The organic solvent used in the method of the invention can be any organic solvent that allows binding of biological molecules, and in this example, binding of nucleic acids, to a substrate. The organic solvent can be, but is not limited to, ethanol, acetonitrile, acetone, tetrahydrofuran, 1,3-dioxolane, morpholine, tetraglyme, dimethyl sulfoxide, and sulfolane. In preferred embodiments, nucleic acids are bound to the substrate in the presence of sulfolane and chaotropic salts. The final concentration of organic solvent may be any amount that allows for binding of the molecule of interest. For nucleic acids, it can range from 0% to 100%, such as from 15% to 80%, for example from 20% to 50%. In embodiments where the target molecule is RNA, a final concentration of about 15% to about 45% (e.g., 35%, 36%, 37%, 38%, 39%, 40%) organic solvent is typically employed for the method to maximize RNA binding. In embodiments where low molecular weight double- and single-stranded nucleic acids are the target molecules, a final concentration of about greater than 40% can be used. While not being limited to any one mode of action, it is envisioned that relatively low concentrations of organic solvent favor binding of RNA, whereas relatively high concentrations of organic solvent permit binding of low molecular weight double- and single-stranded nucleic acids. Preferably, the purity of the organic solvent is about 98% or greater, for example 99.5% or 99.8%.

The method also comprises mechanically combining the sample with chaotropic salts before binding to the mineral substrate. Combining can be any action that results in the sample and salt coming into contact. Combining may be done by adding a lysis buffer comprising high salt to the sample. For isolation of nucleic acids, preferably, the salts in the lysis buffer are chaotropic salts found in a concentration from about 0.1 to about 10 M, such as from about 1 to about 5 M or from about 5 to about 10 M. In a preferred embodiment, 4 to 5 M salt is used in the lysis buffer. The salts used in these methods may be chaotropic salts, such as guanidinium chloride, guanidinium thiocyanate, guanidinium isothiocyanate, sodium perchlorate, and sodium iodide. Non-chaotropic salts include salts of Group I alkali metals, such as sodium chloride, sodium acetate, potassium iodide, lithium chloride, potassium chloride, and rubidium and cesium based salts. As a general matter, any salt that will allow the binding of a biological molecule to the mineral substrate in the presence of organic solvent may be used in this method. The salts in the invention may be one particular salt or may comprise combinations thereof such that a mixture of salts is used. Urea, another chaotropic substance, in concentrations from 0.1 to 10 M may also be used for lysing and/or binding the sources containing the biological molecules. In one embodiment, sarkosyl (preferably 0.05%) is added to the lysis buffer to reduce double-stranded nucleic acid content when single-stranded nucleic acid is being isolated and possibly to reduce RNase activity.

The mineral substrate used for adsorbing a nucleic acid molecule is preferably a filter that comprises or consists of porous or non-porous metal oxides or mixed metal oxides, silica gel, sand, diatomaceous earth, materials predominantly consisting of glass, such as unmodified glass particles, powdered glass, quartz, alumina, zeolites, titanium dioxide, and zirconium dioxide. Fiber filters comprised of glass or any other material that can be molded into a fiber filter may be employed in this method. If alkaline earth metals are used in the mineral substrate, they may be bound by ethylenediaminetetraacetic acid (EDTA) or EGTA, and a sarcosinate may be used as a wetting, washing, or dispersing agent. Any of the materials used for the mineral substrate may also be engineered to have magnetic properties. The particle size of the mineral substrate is preferably from 0.1 um to 1000 um, and the pore size is preferably from 2 to 1000 um. The mineral substrate may be found loose, in filter layers made of glass, quartz, or ceramics, in membranes in which silica gel is arranged, in particles, in fibers, in fabrics of quartz and glass wool, in latex particles, or in frit materials such as polyethylene, polypropylene, and polyvinylidene fluoride. The mineral substrate may be in the form of a solid such as a powder or it may be in a suspension of solid and liquid when it is combined with a liquid sample. The mineral substrate can also be found in layers wherein one or more layers are used together to adsorb the sample.

The present invention can also be utilized to selectively bind either a single-stranded or double-stranded nucleic acid to a mineral substrate. Nucleic acid binding to the mineral substrate is a function of the amount of organic solvent and salts present during binding, among other factors. Under certain conditions of salt and where organic solvent concentrations are high (for example, at approximately 30% organic solvent), both types of nucleic acid (DNA and RNA) bind to the mineral support. Under other conditions, where the organic solvent and/or salt concentrations becomes less than a defined value, none of the nucleic acids will bind to the mineral support to any substantial extent. However, in between these two conditions, RNA and DNA will bind to the mineral support to a different extent and thus, the concentrations of salts and organic solvent can be adjusted to selectively bind predominantly one nucleic acid. This method, therefore, is a way to separate nucleic acids by differential binding of DNA and RNA in the presence of organic solvent and salts. In one embodiment, DNA is selectively bound to the mineral substrate under conditions of lower concentrations of organic solvent (e.g., ≦20% organic solvent by volume) and the RNA molecules predominantly flow through. Additional organic solvent can be added to the flow-through fraction containing predominantly RNA from the first mineral substrate, thereby allowing the RNA to bind to a second mineral support. For example, the organic solvent concentration can be raised to ≧30% or more to effect RNA binding. Other variations can be envisioned and utilized to take advantage of the differential binding of the nucleic acids to a mineral substrate in the presence of organic solvent and salts. The automated system of the invention can be configured to fit different variations of the method.

Thus, in embodiments, the invention provides an automated process for the separation of single-stranded nucleic acids from double-stranded nucleic acids by treatment of a biological source, where the treatment comprises: a) applying by mechanical means to a first mineral support an aqueous sample comprising material of the source under conditions whereby the first mineral support adsorbs or binds only one of the single- or double-stranded nucleic acids followed by, optionally, washing the first mineral support; and b) applying by mechanical means to a second mineral support the other of the single- or double-stranded nucleic acids, which was not adsorbed or bound by the first mineral support, in an aqueous solution containing organic solvent. In the process, the applying step to the first mineral support can comprise adding to the aqueous sample salts and organic solvent in amounts such that the single-stranded, but not the double stranded, nucleic acids are adsorbed on or bound to the first mineral support, followed by, optionally, washing of the first mineral support. In addition, the double-stranded nucleic acids, which were not adsorbed on or bound to the first mineral support, can be applied to the second mineral support in the presence of appropriate amounts of one or more salts and organic solvent such that the double-stranded nucleic acids are adsorbed on or bound to the second mineral support, followed by, optionally, washing of the second mineral support. Further, the single-stranded nucleic acids, double-stranded nucleic acids, or both can be eluted from the first and second mineral supports, respectively. According to the process, the applying step to the first mineral support can comprise adding the aqueous sample to materials that complex alkaline-earth metal ions, in the absence of organic solvent, such that double-stranded, but not single-stranded nucleic acids are adsorbed on or bound to the first mineral support. The single-stranded nucleic acids, which were not adsorbed on or bound to said first mineral support, can be applied to the second mineral support in the presence of salts and organic solvent in amounts such that the single-stranded nucleic acids are adsorbed on or bound to the second mineral support, followed by optionally, washing of the second mineral support. Further, the double-stranded nucleic acids, single-stranded nucleic acids, or both can be eluted from the first and second mineral supports, respectively.

In some instances, the process can be characterized by the applying step to the first mineral support comprising adding to the aqueous sample wetting, washing, or dispersing agents, in the absence of organic solvent, such that the double-stranded nucleic acids are adsorbed on or bound to the first mineral support, followed by, washing of the first mineral support. In addition, the single-stranded nucleic acids, which were not adsorbed on or bound to the first mineral support, can be applied to the second mineral support in the presence of organic solvent in amounts such that the single-stranded nucleic acids are adsorbed on or bound to the second mineral support, followed by optionally, washing of the second mineral support. Further, the single-stranded, double-stranded nucleic acids, or both can be eluted from the first and second mineral supports, respectively. In some embodiments, the applying step to the first mineral support comprises adding to the aqueous sample salts and organic solvent in amounts such that both the single-stranded and double-stranded nucleic acids are adsorbed on or bound to the first mineral support, one of the single- or double-stranded nucleic acids is, selectively, first eluted from the first mineral support, followed by eluting the other of the single- or double-stranded nucleic acids, and the one of the single- or double-stranded nucleic acids, which was first eluted from the first mineral support, is applied to the second mineral support under conditions whereby the nucleic acids first eluted from the first mineral support are adsorbed on or bound to the second mineral support, followed by eluting the nucleic acids from the second mineral support.

Once at least one biological molecule has been adsorbed to the mineral substrate, the substrate can be optionally washed with one or more solutions that contain an organic solvent, such as ethanol, an organic solvent similar to ethanol, or mixtures thereof. An organic solvent similar to ethanol means a solvent of “like” chemical and physical properties. For example, the solvent may have similar specific gravity, miscibility in water, or other characteristics that allow it to be a component of the wash buffer without removing the biological molecule from the mineral substrate. “Mixtures thereof” means that more than one kind of organic solvent may be used in the wash buffer. For example, a mixture of ethanol and dioxolane, a mixture of sulfolane and dioxolane, a mixture of ethanol, dioxolane, and acetonitrile, etc. may be used for washing the mineral substrate. There are many variations of mixtures of organic solvents that can be used for this step and the mixture may comprise more than two organic solvents. Optionally, the solution also contains one or more salts. If salt is used in the wash solutions, the salt may be a chaotropic salt or a salt comprising an alkaline metal (e.g., a Group I metal, such as sodium chloride) or alkaline earth metals (e.g., a Group II metal salt). The salt concentration can range from 0.001 M to 3 M. Likewise, the organic solvent may range from a final concentration of 1% or less to 100%, by volume. For example, the organic solvent may be present in a final concentration of approximately 50% by volume. Thus, the range of salt and ethanol and/or other solvent concentrations in the salt solution can be from no salt and 100% ethanol and/or other solvent to 3 M salt and about 80% ethanol and/or other solvent or less. In some embodiments, the solution is a high salt buffer comprising one or more organic solvents (e.g., 10-90% by volume) and having a salt content of about 50 mM or greater. In other embodiments, the solution is a low salt buffer comprising one or more organic solvents (e.g., 10-90% by volume) and having a salt content of less than about 50 mM, such as one comprising 20 mM NaCl and from about 50% to about 60% ethanol (e.g., about 52%, 54%, 56%, 58%). Methods of washing are well known in the art (such as adding the buffer to the sample and then centrifuging the sample or applying positive air pressure and/or vacuum to the sample) and therefore will not be described in detail herein. Any suitable washing scheme may be used. Where high salt and low salt washing buffers are used, it is preferable that the high salt wash be performed first, as a goal of the washing is to remove unwanted biological materials, followed by the low salt wash to reduce the amount of salt associated with the bound material.

Thus, before elution of the biological molecules from the mineral substrate, the substrate can be treated with one or more high salt washes to remove contaminating proteins, including DNase or RNase. The high salt wash is comprised of, for example, 1 to 8 M salt and 20% to 80% ethanol or other organic solvent or mixture of solvents. In a preferred embodiment, the high salt wash is comprised of 2 M chaotropic salt and about 50% to about 60% ethanol. This optional high salt wash step can incorporate one or more high salt washes. In a preferred embodiment, when RNA is being isolated and a DNase step is used, two or three high salt washes are performed comprising 2 M guanidinium thiocyanate and about 50% to about 60% ethanol or solvents of “like” physical and chemical properties. Where desired, a low salt solution, such as that described above, can be used after the high salt washes to lower the salt concentration of the nucleic acid containing composition.

After the optional first and/or second washes, the mineral substrate can be treated with DNase, RNase, proteases, or other enzymes in an appropriate aqueous environment to remove biological compounds that are not of interest. In one preferred embodiment, RNA is the biological molecule of interest and a DNase digestion buffer is added to eliminate DNA molecules from the mineral substrate. In another embodiment, DNA is the molecule of interest and an RNase digestion buffer is added to eliminate RNA molecules from the mineral substrate. Following DNase or RNase treatment, the mineral support is washed with high salt and low salt washing buffers, respectively, to remove residual DNase or RNase and salts. When the method is automated, specific computer programs can be established depending on the biological molecule or cell of interest.

The step of eluting the biological molecules from the mineral substrate can comprise first drying (e.g, by passing air over the solid phase substrate) the mineral substrate to eliminate water and the organic solvent (e.g., ethanol), then adding a liquid, such as elution buffer or water, to the substrate, optionally allowing the liquid to incubate with the substrate from zero to one hour or more, and separating the liquid from the substrate. Under some circumstances, the bound biological molecules can be exposed to a highly volatile organic compound, such as acetone, to facilitate removal of water and other organic compounds by evaporation. In embodiments where nucleic acids are being eluted, incubation typically can occur from about zero seconds to about 20 minutes, such as from about zero seconds to about 10 minutes, or from about zero to about 5 minutes. In a preferred embodiment, incubation occurs for about 2 minutes. During this step, most of the nucleic acid molecules bound to the substrate should elute into the liquid. Incubation can occur with a liquid that is warm, such as from about 26° C. to about 80° C. or close to room temperature, such as from about 20° C. to about 25° C. Preferably, where the elution solution (e.g., buffer) comprises salts, the salts have a pKa value from about 6 to about 10 and the buffer has a salt concentration up to about 100 mM. For example, 10 mM Tris (pKa 8.0) pH 8.5 may be used to elute the biological molecule from the mineral substrate. Elution may occur in one step or may be done using several elution steps. The instrument may be programmed to allow variable numbers of elution steps.

In embodiments relating to purifying RNA, the bound RNA may be eluted from the second solid phase substrate by eluting with water or a low salt solution, such as a buffer. The eluted RNA is collected in the substance collection port. Alternatively, once the RNA is bound to the second solid support, the entire purification cartridge can be removed from the instrument and further processed for isolation of the RNA. For example, the entire purification cartridge can be sent to a processing facility for elution and analysis. Optionally, the cartridge can be stored for indefinite periods of time prior to analysis. Alternatively, the solid support to which the RNA is bound can be removed from the cartridge, optionally stored, and used for analysis.

As mentioned above, in the method or process of the invention, salts can be present in concentrations of from 1 to 10 M. For example, the process or method can comprise, prior to applying a sample to a first mineral support, lysing cells in a source containing the nucleic acids with chaotropic substances present in concentrations of from 0.1 to 10 M. To reiterate, in the processes and methods of the invention, organic solvent can be present in concentrations of from 1 to 90% by volume or more, final concentration. In addition, the make-up of the first and second mineral supports is not particularly limited, and thus can be, independently, for example, porous or non-porous and comprised of metal oxides or mixed metal oxides, silica gel, glass particles, powdered glass, quartz, alumina, zeolites, titanium dioxide, or zirconium dioxide. The particle size of the mineral supports is likewise not limited, and can be, for example, from 0.1 micrometers to 1000 micrometers. Further, the pore size of porous mineral supports is not limited, and can be, for example, from 2 to 1000 micrometers. Complexes formed in the process can comprise alkaline earth metal ions bound to ethylenediaminetetraacetic acid (EDTA) or EGTA. Furthermore, where a wetting, washing, or dispersing agent is used in one or more lysing, binding, or washing solutions, the wetting, washing or dispersing agent can be a sarcosinate.

After elution of the biological molecules from the mineral substrate, the isolated biological molecules can be removed from the machine. “Removed” as described herein means that the sample can be manually removed or can be taken from the system in an automated fashion. If RNA is the isolated biological molecule, it is directly suitable for assays such as RT-PCR, microarrays, etc. Thus, further actions on the isolated biomolecule may be performed, such as analysis of the biomolecule for purity, size, or chemical constituency (e.g., nucleic acid sequence, by way of sequencing or hybridization to nucleic acids of known sequence). Alternatively, the instrument can contain the required components to further manipulate or assay the isolated biological molecules. For example, a component of the system may be able to dilute the sample by adding a buffer to the eluted nucleic acid. A further step may be to concentrate the biological molecules or cells using more filters or adding an ethanol precipitation step. Yet another further step may allow the biological molecules or cells to be assayed directly as part of the method. For example, isolated RNA may be assayed by RT-PCR directly in the system. As another example, mRNA may be separated from the total RNA that has been isolated directly in the same machine. Components can be added to the instrument to further manipulate the biological molecules or cells of interest.

The method of the present invention comprises exposing one or more biological molecules to an organic solvent and a mineral support for a sufficient amount of time for some or all of the biological molecules to be adsorbed or otherwise bound to the mineral support. The biological molecule of interest may be one bound to the mineral support, or one found in the un-bound fraction. For example, in a composition comprising a nucleic acid and a protein, the nucleic acid may be bound to the mineral support under the described conditions, whereas the protein may remain unbound. In this way, both molecules may be purified away from each other. Subsequently, salts, buffers, solvents, etc. can be added to the optimal conditions for purification of a variety of protein species employing filters, resins, etc. The method may also comprise exposing the biological molecule to one or more salts, such as chaotropic salts. The method may also comprise removing the organic solvent, salts, and/or any unbound substances, by washing the mineral support and bound material, and/or releasing the bound material from the mineral support.

In another embodiment, the method of the invention comprises the isolation of a specific protein from a biological sample. In this case, salts that may or may not be chaotropic can be used in conjunction with an organic solvent to bind nucleic acids to at least one mineral support. Under these conditions, proteins will not bind to any appreciable extent, and can thus be captured in flow-through or eluate fractions, free or essentially free of one or more nucleic acids. For some proteins and protein analysis techniques (e.g., enzyme activity assays), the conditions for binding should be such that the protein of interest is not denatured or otherwise non-reversibly altered in tertiary or quaternary structure. However, for some proteins, denaturation is acceptable if renaturation may be accomplished without significant detriment to the structure or activity of the protein. Also, compositions comprising proteins that do not bind to a mineral support in the presence of organic solvent, alone or in the presence of one or more salts, can be exposed to the mineral support so that DNA and/or RNA is adsorbed. The protein of interest will flow through and can then be purified using protein purification methods known to those of skill in the art (for example, using ion exchange chromatography, hydrophobic interaction chromatography, gel filtration, affinity chromatography, etc.). As an example of proteins that may be of interest in blood, antibodies (immunoglobulins), Factor VIII, albumin, fibrinogen, etc. may be separated from nucleic acids using this method. As in the other embodiments, the steps for isolation of the specific protein can be performed by a machine and therefore can be fully automated. The system may separate the specific protein from other types of biological molecules, such as nucleic acids. In addition, the machine may also contain components that further manipulate the specific protein, such as protein purification columns that allow further separation of the specific protein from other proteins.

In still another embodiment, the method of the present invention is a way to purify other blood components. For example, after binding of blood cells to the prefilter, the flow-through may contain blood serum or plasma that has been partially purified. The method can be set up so that further components of the system allow still more purification of the blood serum, blood plasma or other components of blood. The method may involve further capture of other undesirable blood components so that the flow-through is the desired fraction. The process may also involve further capture of desired components in the blood that can be isolated using other filters, columns etc. For example, depending on the pore size of the prefilter and the mineral substrate used in the method, platelets in the blood may be captured on either the prefilter or the mineral substrate. If the platelets flow through both of these filters, additional filters can be added to the method of the present invention to adsorb the platelets in a further step. By this method, platelets may be separated from red blood cells, white blood cells, blood proteins, and/or blood plasma. The method of the invention can be varied so that different components of the blood may be separated from other components, depending on the goal of the method.

The methods of the present invention can be fully automated, such that all of the steps are performed by a machine or instrument, with the exception of the addition or removal of the sample from the instrument which may or may not be automated. Components of the system, which can be varied depending on the goal of the method, allow the method to occur without any pretreatment of the samples. For example, whole blood or cell cultures can be added to the system without any dilution, addition of buffer, or any other pretreatment. This not only saves the time of the user but also allows more reproducibility to the method. There are many other advantages to automating the methods, only some of which are delineated herein. For example, the system is closed so the purification of the biological molecules or cells occurs without additional contamination from environmental sources. In the case of isolation of RNA molecules, this minimizes RNases from human hands and particles in the air from entering the isolation chambers. Because the method is relatively quick, with purification of some molecules occurring in 15 minutes or less, the chances of degradation of the molecules or cells of interest is minimized.

The methods of the present invention are implemented via computer programs. The instrument can have computer programs already preprogrammed into it and/or the user can program custom methods into the system. Different programs can be added to the instrument depending on the method for isolation. Computer programs already installed in the machine can be changed to reflect different methods and goals. For example, an automated program can delineate the steps for purification of RNA molecules from whole blood. In this case, the method of the invention will include specific steps required to isolate RNA molecules. Another computer program can incorporate the process of genomic DNA isolation from cultured cells. In this case, the method of the invention will include specific steps required for the purification of large DNA molecules. The computer programs can be set up in such a way that not all chambers of the system are employed in a method or all the chambers are used for the specific isolation. In some embodiments, parts of the instrument can be taken out and exchanged for another part that is better suited for a specific method. This may involve removal of whole chambers or removal of smaller parts of the system, such as a filter, column, tubes, etc.

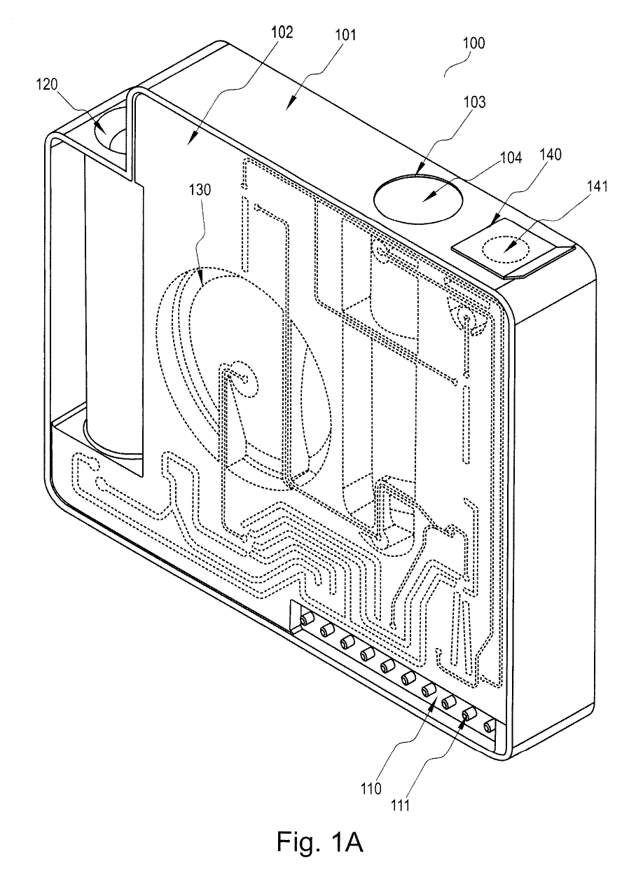

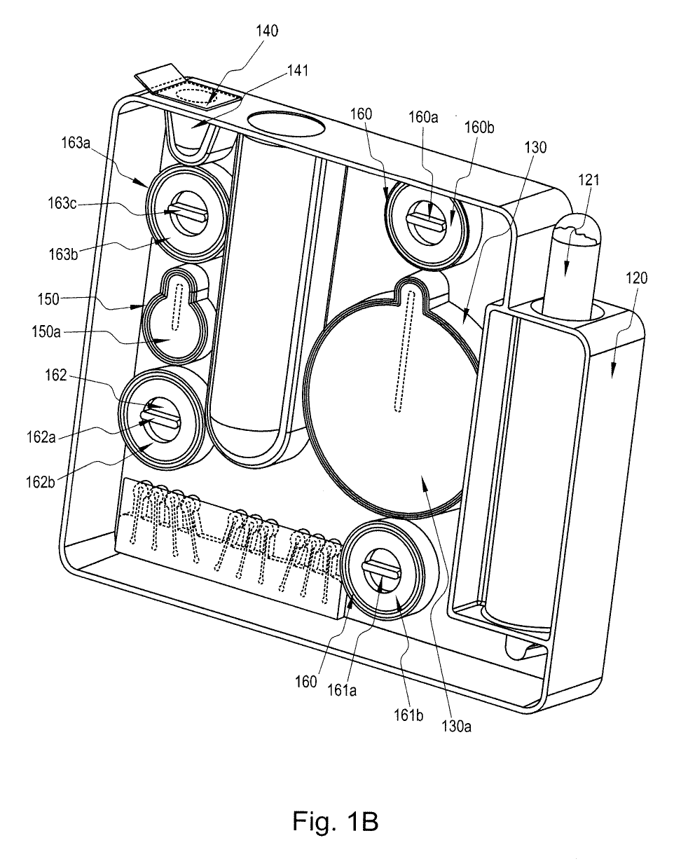

Turning now to the figures, which depict certain embodiments of the purification cartridge of the system, one can see in FIG. 1 a purification cartridge 100 according to one embodiment of the invention. FIG. 1A depicts the exemplary cartridge from a front perspective. As can be seen in this panel, cartridge 100 comprises an outer shell 101 and a front cover 102. Disposed in the surface of outer shell 101 is a sample receiving zone or port 120, a mixing chamber port 103 defined at the surface of outer shell 101 by reversibly attached cap 104, and a collection chamber port 140 defined at the surface of outer shell 101 by hinged cap 141. Entry port interface 110 comprises multiple inlet and outlet ports 111 disposed on front cover 102. Within the context of the system as a whole, ports 111 function for entry of various fluids into the cartridge from a reagent pack (not depicted) and removal from the cartridge waste substances (in embodiments these are transported into the reagent pack; in embodiments, these are transported to a separate waste container or to the environment). Pre-filtration zone 130 is disposed within front cover 102.

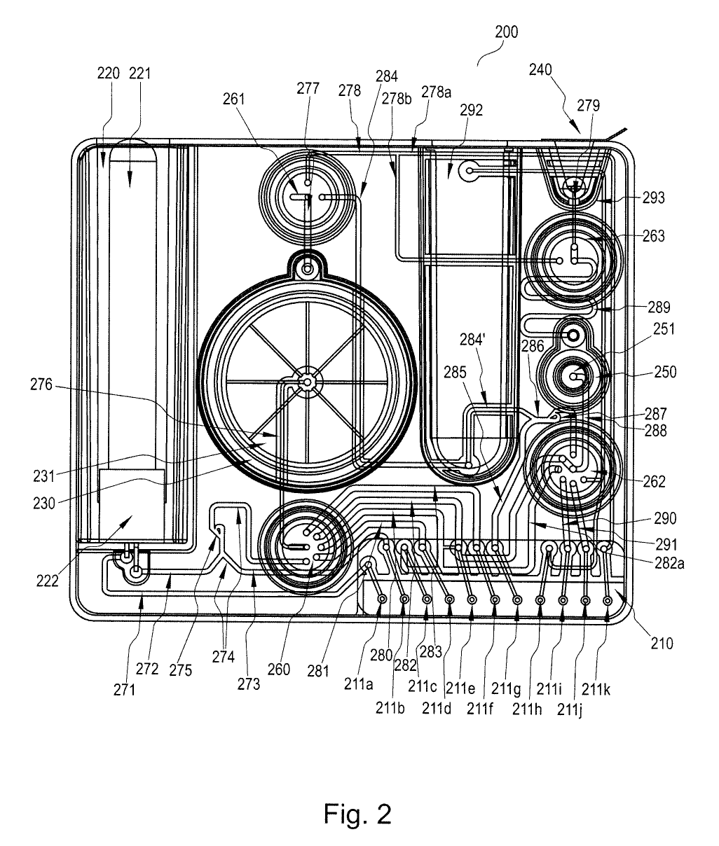

FIG. 1B depicts the exemplary cartridge from a rear perspective. As can be seen, sample receiving zone 120 comprises sample container 121. Rotary valves 160, 161, 162, and 163 are depicted with conduits 160a, 161a, 162a, and 163a and sealing rings 160b, 161b, 162b, and 163b, respectively. Pre-filtration zone 130 is defined on the outer surface by zone cover 130a. Second filtration zone 150 defined on the outer surface by zone cover 150a are shown, as is substance collection port 140 and cap 141. As can be seen in FIG. 1B, each of valves 160, 161, 162, and 163 comprise a conduit (160a, 161a, 162a, and 163a, respectively) that allows fluid from one conduit that is connected to the valve to flow to another conduit connected to the valve. Rotation of the valve causes conduits 160a, 161a, 162a, and 163a to connect different conduits to each other. Various conduits are also depicted, as described in detail below with reference to FIG. 2. The cartridge may be of any size suitable for use in a purification scheme. In embodiments, it measures approximately 5.75 inches by 5 inches by 1 inch.

FIG. 2 depicts a cross-section of the cartridge of FIG. 1, showing details of the conduits and showing various filtration units and valves of the cartridge. More specifically, FIG. 2 depicts a cartridge 200 comprising an entry port interface 210, a sample receiving zone 220 comprising a blood sample container (e.g., blood collection tube or vial) 221, a pre-filter zone 230 comprising a pre-filter filtration unit 231. Cartridge 200 further comprises a second filtration unit zone 250 comprising a second filtration unit 251, and a substance collection port 240. Four rotary valves, 260, 261, 262, and 263, are depicted as well, as are various conduits connecting these elements, as described in detail below.

Returning now to FIGS. 1-2 in combination, operation of the cartridge for purification of RNA from white blood cells is described in detail. It is to be noted that reference will be made to elements depicted in FIG. 2; however, like elements are depicted in FIG. 1 with similar numbering and elements. Initially, conduits may be “primed” with various fluids by opening or closing valves 260, 261, 262, and 263 as needed to allow an open circuit between an inlet port 211a-j and exit port 211k. Furthermore, by applying a vacuum to exit port 211k, various fluids may be drawn or pulled through appropriate conduits. This pulling action may supplement or replace the positive force applied through inlet ports 211a-j.

To purify RNA from a blood sample, such as for example a 5 ml whole blood sample, blood sample container 221 is inserted into cartridge 200 by way of sliding into sample receiving zone 220. Full insertion into sample receiving zone 220 causes puncture of blood sample container at cap 222 by two needles (not shown). Air is caused to flow through air intake port 211a, through conduit 271, and into blood sample container 221 by way of a needle (not shown), resulting in pressurization of blood sample container 221. Blood in blood sample container 221 flows from container 221 into conduit 272 by way of a needle (not shown). Concurrently, red blood cell (RBC) lysis solution is caused to flow through RBC lysis solution intake port 211b and through conduit 273. Blood and RBC lysis solution (e.g., an equal volume of each) are combined at the juncture of conduits 272 and 273, causing mixing of the two compositions in conduit 274 and lysis of red blood cells. Further mixing of the two compositions and further lysis of red blood cells is accomplished by channeling the combination of solutions through a first static or Tesla mixing chamber 275. The mixture flows from mixing chamber 275 through conduit 274 and into rotary valve 260. Rotary valve 260 is caused by a computer controlled actuator (not shown) to rotate such that conduit 260a forms a connection between conduit 274 and conduit 276. The mixture is caused to flow into pre-filtration zone 230, entering the zone by way of a port in the center of the proximal side of the zone. The mixture contacts pre-filtration unit 231 and travels down filtration cone 232 by way of cone channels 233 to the perimeter of filtration zone 230. The mixture then proceeds over a filtration unit (not depicted), which entraps unlysed cells, including white blood cells and unlysed red blood cells. Filtered fluid (eluate) exits filtration zone 230 by way of an exit port in the center of the distal side of the zone (not depicted), and travels by way of conduit 277 to valve 261.

Where the unbound material (eluate) is to be discarded, valve 261 is actuated by a computer controlled actuator (not depicted) such that conduit 261a forms a connection between conduit 277 and conduit 278. Eluate is caused to exit cartridge 200 via waste conduit 278a through waste exit port 211k. Where the eluate is to be saved, exit port 211k is caused to be closed (e.g., by providing back-pressure that blocks movement of fluids through conduit 278a). Blocking of conduit 278a causes eluate to enter conduit 278b and enter valve 263. Valve 263 is actuated to cause conduit 263a to connect conduit 278b and conduit 279, and eluate is caused to enter substance collection port 240, where it may be removed by the user.

After passing the blood/RBC lysis mixture over pre-filtration unit 231, pre-filtration unit 231 may be exposed to additional RBC lysis mixture (e.g., an equal volume, two volumes, etc.) to improve the total lysis of red blood cells, and preferably cause essentially total red blood cell lysis. To do so, red blood cell lysis solution is caused to enter cartridge 200 through inlet port 211b. RBC lysis solution flows through conduits 273 and 274 to valve 260. Conduit 260a is rotated to create a fluid connection between conduit 274 and 276, and RBC lysis solution is passed over pre-filtration unit 231 as described above. Passing of RBC lysis solution over pre-filtration unit 231 causes lysis of RBC entrapped by the pre-filtration unit. Waste RBC lysis solution, cell debris, and other materials are either discarded to waste or captured, as described above.

At this juncture, air may be caused to enter cartridge via intake port 211c, and flow through conduit 280 to valve 260. Valve 260 may be actuated to cause conduit 260a to connect conduits 280 and 276, resulting air flowing over pre-filtration unit 231 and exiting cartridge 200 by way of conduits 277, 361a, 278, 278a and port 211k, or by way of conduits 277, 361a, 278, 278b, 263a, 279, and port 240. It is to be noted that, if desired, air may also have been or concurrently be caused to flow through conduits 271 and 272 to remove residual fluids in those conduits, and the air caused to flow out of cartridge 200 as described above from valve 260.

Optionally, pre-filtration unit 231 can be washed with phosphate-buffered saline (PBS) before or after the optional air purge of conduits. PBS, for example 12.5 ml at 300 microliters per second, is caused to enter cartridge 200 by way of intake port 211d. PBS flows through conduit 281 to valve 260. Valve 260 is actuated to align conduit 260a with conduits 280 and 276. PBS is caused to flow through conduit 276 over pre-filtration unit 231 and out of pre-filtration zone 230 through conduit 277. The PBS may then exit cartridge 200 as described above with regard to eluate. If desired, pre-filtration unit 231 may be washed one or more additional times, for example by flushing with PBS at 600 microliters per second. At this time, a second optional air drying step may be performed, as described above.

Additionally, water can be used to wash pre-filtration unit 231. In doing so, water is caused to enter cartridge 200 by way of intake port 211e. It is caused to flow through conduit 282 to valve 260. Water is then caused to flow over pre-filtration unit 231 and out of cartridge 200 as described above with regard to PBS.

After causing RBC lysis solution to flow over pre-filtration unit 231 and any optional washes and conduit purges, white blood cells entrapped by the unit are lysed by causing white blood cell (WBC) lysis solution to pass over pre-filtration unit 231. WBC lysis solution is caused to enter cartridge 200 by way of intake port 211f. WBC lysis solution is caused to travel through conduit 283 to valve 260. Valve 260 is actuated such that conduit 360a causes a fluid connection between conduits 283 and 276, and WBC lysis solution is caused to pass over pre-filtration unit 231 and out of pre-filtration zone 230 as described above for other solutions. Passing of WBC lysis solution over pre-filtration unit 231 causes lysis of WBC entrapped by the unit. For example, 9 ml of WBC lysis solution may be passed over the pre-filtration unit at 400 microliters per second to cause WBC lysis. Cell debris and large nucleic acids are entrapped by pre-filtration unit 231, whereas small molecules, including RNA, flow through. The flow through fluid passes through conduit 277 to valve 261. Valve 261 is actuated such that conduit 261a connects conduit 277 to conduit 284. Eluate from cell lysis, which includes RNA, is caused to travel through conduit 284 and enter mixing chamber 292, where it is collected. During or immediately after passing the WBC lysis solution over pre-filtration unit 231, the flow of fluid can be paused for a period of time to allow for increased cell lysis. For example, the flow of fluid may be paused for from about one second to about ten minutes or more. Preferably, pausing is kept relatively short, such as, for example, less than or about three minutes, less than or about two minutes, or less than or about one minute. After the optional pause, additional WBC lysis solution is caused to pass over pre-filtration unit 231. For example, an additional 5 ml may be passed over the pre-filtration unit. The additional WBC lysis solution is collected in mixing chamber 292 as described above. Of course, as with other steps, before or after conducting the WBC lysis, one or more conduits may be purged of fluids by causing air to flow through the conduits. Furthermore, where desired, purge air may be caused to run through mixing chamber 292 prior to exiting cartridge 200 to improve mixing of the liquid composition contained in chamber 292.

To the RNA-containing composition maintained in mixing chamber 292, water may be added to alter the volume and/or adjust the concentrations of certain substances in the composition. To do so, water may be caused to enter mixing chamber 292 by way of entry port 211e and flow over pre-filtration unit 231, as described above from valve 260. For example, 4 ml of water may be added to the mixing chamber. It is to be noted that this step further washes pre-filtration unit 231 and improves RNA yield.

The RNA-containing mixture is then caused to flow over second filtration unit 251 by causing air to be introduced into mixing chamber 292. More specifically, air can be introduced into cartridge 200 through inlet port 211h and conduit 293. Concurrently, a solution comprising sulfolane or another organic solvent is caused to enter cartridge 200 by way of intake port 211g. It is caused to flow through conduit 285. Meanwhile, the RNA-containing solution is caused to exit mixing chamber 292 via conduit 284′. Conduits 284′ and 285 merge to form conduit 286, where mixing of the sulfolane and the flow-through occurs. For example, an equal volume of 80% sulfolane may be mixed in the conduits with the RNA-containing composition flowing from mixing chamber 292. Further mixing of the two compositions is accomplished by channeling the combination of solutions through a second static or Tesla mixing chamber 287. The mixture flows from mixing chamber 287 through conduit 286 and into rotary valve 262. Rotary valve 262 is caused by a computer controlled actuator (not shown) to rotate such that conduit 362a forms a connection between conduit 286 and conduit 288. The mixture is caused to flow through conduit 288 into second filtration zone 250, which comprises second filtration unit 251. RNA present in the mixture binds to filtration unit 251, and unbound material is caused to exit filtration zone 250 by way of conduit 289. Eluate from the second filtration unit 251 is caused to pass through conduit 289 to valve 263.

Where the unbound material (eluate) is to be discarded, valve 263 is actuated by a computer controlled actuator (not depicted) such that conduit 263a forms a connection between conduit 289 and conduit 278b. Eluate is caused to exit cartridge 200 via waste conduit 278a through waste exit port 211k by closing valve 261. Where the eluate is to be saved, valve 263 is actuated by a computer controlled actuator (not depicted) such that conduit 263a forms a connection between conduit 289 and conduit 279, causing eluate to enter substance collection port 240, where it may be removed by the user.

Passing the RNA/sulfolane mixture over second filtration unit 251 causes RNA to bind to the filtration unit. At this juncture, purging of conduits and drying of filtration unit 251 with air may be performed by introducing air into the cartridge via intake port 211h and conduit 293, through mixing chamber 292 and through conduits 284′, 286, valve 262/conduit 262a, and conduit 288.

The RNA bound to filtration unit 251 may be washed with one or more appropriate substances. In this example, filtration unit 251 is washed with a low salt buffer, which is introduced into cartridge 200 by way of intake port 211i. Low salt wash buffer, for example 2.5 ml at 400 microliters per second, is caused to travel through conduit 290 to valve 262. Valve 262 is actuated such that conduit 262a aligns with conduits 290 and 288 and allows fluid to move to filtration unit 251. Flow-through from filtration unit is caused to either be saved by way of collection port 240 or caused to be discarded as waste through conduits 278b and 278a, as discussed above. Air may be used to dry the conduits and second filtration unit 251, if desired, by causing air from inlet port 211h to travel through conduit 293, mixing chamber 292, and over filtration unit 251, as described above. If desired, one or more subsequent low salt washes (e.g., with 2.5 ml of low salt buffer) may be performed, with optional air purges in between. A final air purge may be performed with a relatively long cycle time to improve drying.

The washed filter-bound RNA may be additionally exposed to an ethanol-containing composition, such as, for example 100 microliters or more of absolute ethanol. The ethanol composition is caused to enter cartridge 200 by way of intake port 211j. The ethanol solution is caused to traverse conduit 291 to valve 262. The ethanol solution may then be passed over filtration unit 251 and retained or discarded, as described above. An optional air purge may then be performed, as described above.

At this stage, purified RNA is bound to filtration unit 251. The purified RNA may be maintained on unit 251 for an extended period of time or may be eluted immediately. Where the RNA is to be eluted from unit 251 while cartridge 200 is connected to the remaining elements of a system of an invention, it may be eluted as follows. Water or a low ionic strength buffer may be caused to enter cartridge 200 by way of intake port 211e. The water is caused to flow through conduit 282a to valve 262 by causing valve 260 to be closed. Valve 262 is actuated to cause conduit 262a to form a connection between conduits 282a and 288. Water or low ionic strength buffer, such as 200 microliters of water, is then caused to flow over filtration unit 251, causing release of the bound RNA. Optionally, the water may be allowed to pause while in contact with second filtration unit 251 for a period of time, for example one minute, two minutes, five minutes, etc. Eluted RNA is then caused to flow through conduit 289 to valve 263 by pressure from air intake port 211h, as described above. Valve 263 is actuated to cause conduit 263a to form a link between conduits 289 and 279. The eluted RNA is then caused to enter collection port 240 for removal by the user.

It should be evident that, in order to make certain fluids flow through selected conduits, various valves will need to be opened or closed to allow for pressure in certain conduits to be equalized. Suitable valve openings and closings to effect this pressure stabilization have not be detailed in this description, but will be immediately recognized by those of skill in the art.

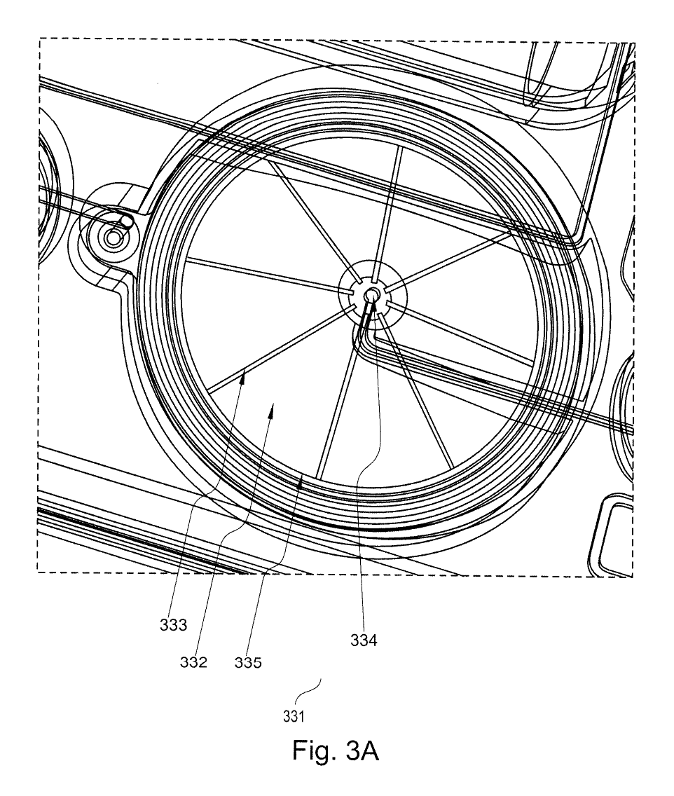

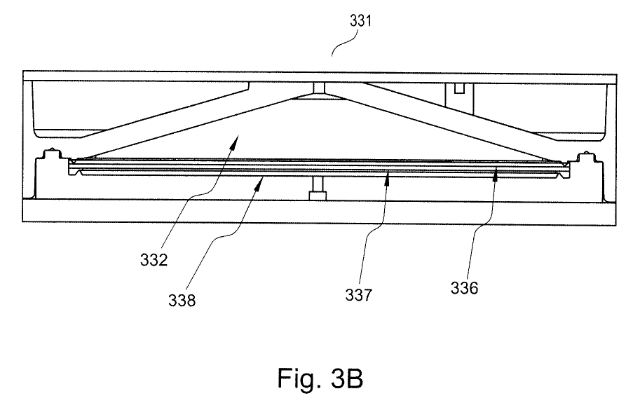

One feature of the cartridge discussed above is the pre-filtration unit. Broadly speaking, this feature comprises one or more solid phase supports for capturing at least some substances in a sample. In the exemplary embody above, the unit is designed to entrap cells, and in particular white blood cells. Various configurations of parts of the unit are possible. One preferred configuration is shown in FIG. 3.

As shown in FIG. 3A, when viewed from the top (also referred to herein as the proximal end), it can be seen that the filtration unit 331 comprises a cone-shaped support or fluid director 332. Disposed within the surface of the director 332 are two or more channels or grooves 333 emanating from a central sample receiving zone 334, which is disposed at the peak or apex of the director 332. In practice, sample is applied to the filtration unit 331 at the apex. Channels 333 cause the sample to flow from the apex down the surface of the director 332 to the perimeter 335. The director 332 is shown in the figure with eight channels; however, it should be understood that any number of channels may be used. Testing of the cone has shown that eight channels provides excellent distribution of sample; however, additional channels should provide similar results.

FIG. 3B depicts a filtration unit 331 in a side view as disposed in a chamber or reservoir of a cartridge. In use, the parts are in direct contact with each other as would be expected from compression of the parts in a proximal to distal plane in their currently depicted state. Director 332 is depicted at the top or proximal side of the unit. Immediately distal to director 332 is screen or mesh 336, which can be included to filter out large particulate matter from samples prior to the sample contacting other components. Screen 336 also may be used to assist in movement of sample from the perimeter of the mesh to the central portion. Immediately distal to screen 336 is solid support or filter 337. Solid support 337 may be any of the materials discussed herein. It should be noted that FIG. 3 depicts a single solid support; however, this element may comprise, in embodiments, multiple individual solid support elements that are combined into a single functional unit. Immediately distal to solid support 336 is screen or mesh 338. As with screen 336, screen 338 may be used to filter out large particles, particularly to ensure that conduits of the cartridge, which may have relatively small diameters, will not become plugged with sample material. However, it is envisioned that the principal function of screen 338 is to provide mechanical support to filter 337 between the filter and the wall of the cartridge.

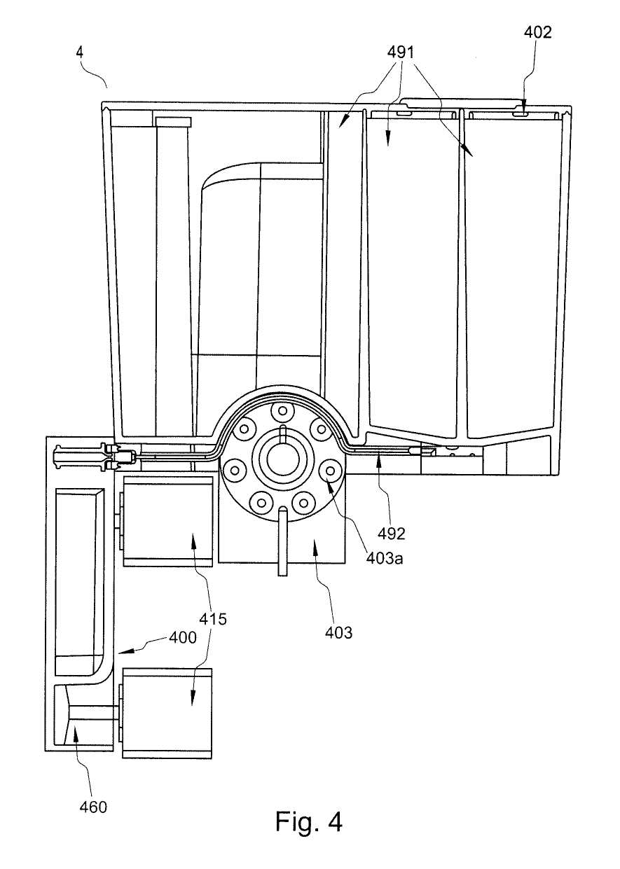

FIG. 4 depicts an embodiment of the system of the invention, showing schematically one configuration of the system 4. The figure depicts system 4 without depicting the shell or outer housing of the instrument. Likewise, motors and linkages for driving actuators are not depicted, nor is a computing device and other optional elements.

The figure shows that a reagent pack 402 comprises multiple containers 491 for containing reagents and solutions, each of which is connected to a flexible tube 492 for movement of fluids from the containers 491 to cartridge 400. As can be seen, cartridge 400 mates with reagent pack 402 to form a fluid-tight seal at each of tubes 492 for movement of fluids from reagent pack 402 to cartridge 400. Movement is effected by peristaltic pump 403, which contacts tubes 492 at pump head 403a. Purification cartridge 400 can have one or more ports for intake and exit of fluids, and each port may comprise a connector for connecting to tubing 492 of or attached to reagent pack 491, such as a male connector or nipple which inserts into the end of tubing 492 to create a seal. In a similar manner, tubing 492 may have, on its other end, a male connector which may insert into a receptacle on containers 491, for example to pierce a foil seal that serves as a partial surface for containers 491, thus allowing fluid to flow from containers 491 into tubing 492.

Cartridge 400 has attached to it motors and electronics units 415, which function to drive rotary valves 460 on cartridge 400. It is to be noted that all valves for the purification system that regulate flow of fluids during the purification process are located on cartridge 400. Motors 415 are reversibly attached to cartridge 400, allowing for ease of replacement of cartridge 400 and a reduction in expense.

Motors 415 are mounted to the shell of the instrument (not depicted), and are controlled from inside the instrument by electromechanical means known in the art. Reagent pack 402 and cartridge 400 are likewise attached to the housing of the instrument. The instrument contains one or more pumps 403 for movement of fluids within the system. System 4 further comprises a computing device (not depicted), which in this embodiment is housed within the instrument.

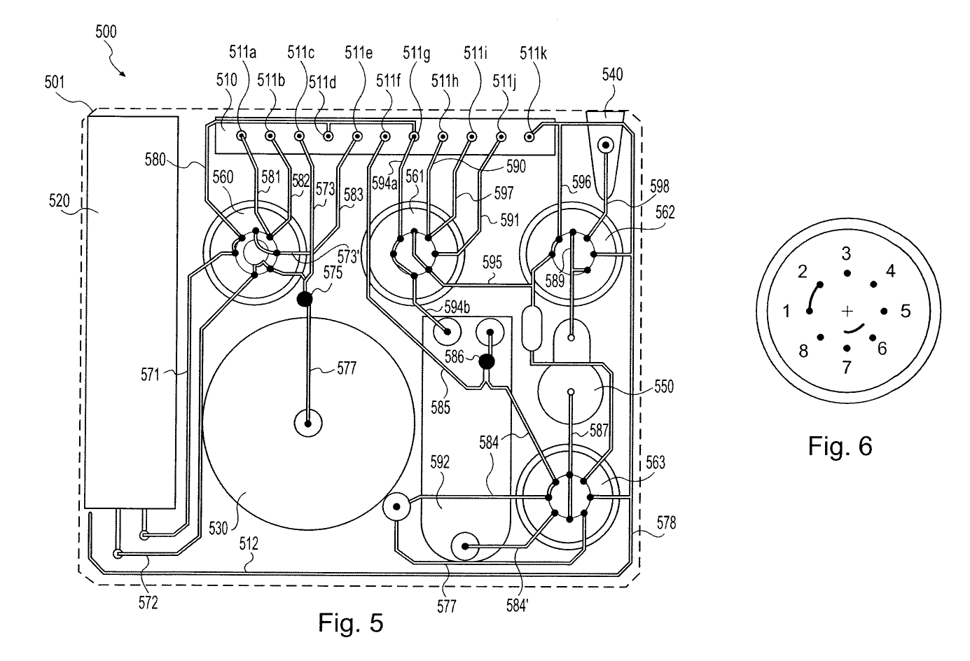

FIG. 5 depicts another exemplary embodiment of a cartridge according to the present invention. In general, the cartridge is approximately 5 inches in height, 6 inches in width, and one inch in depth. Various channels are disposed within the shell of the cartridge, each channel having a width of about 0.03 inches and a depth of about 0.05 inches. The channels may be substantially round to elliptical in cross section to substantially rectangular to square in cross section. Also disposed on the cartridge are valves for controlling flow of fluids through the cartridge. The valves are on the order of about 1 inch in outer diameter.

According to this exemplary embodiment, the cartridge comprises a purification cartridge 500 comprising an outer shell 501 that has disposed within its surface multiple channels, valves, ports, chambers, and mixers. Immediately adjacent to the exterior perimeter of outer shell 501 and following the outer perimeter along a portion of the perimeter is channel 512, which acts as a vacuum trap to contain any possible fluid leaks from breaches in the seal between cartridge outer shell 501 and the front cover of the cartridge (not shown). Channel 512 collects all leaked fluids and channels them to exit port 511k. While not typically used, channel 512 is provided as a fail-safe element to ensure that biohazard materials do not unintentionally leak from the cartridge.

Cartridge 500 also comprises sample receiving zone or port 520, mixing chamber 592, and collection chamber port 540, as well as four rotary valves, 560, 561, 562, and 563. Entry port interface 510 comprises multiple inlet and outlet ports 511 disposed on the bottom surface of outer shell 501. Within the context of the system as a whole, ports 111 function for entry of various fluids into the cartridge from a reagent pack (not depicted) and removal from the cartridge waste substances (in embodiments these are transported into the reagent pack; in embodiments, these are transported to a separate waste container or to the environment). As shown in the figure, cartridge 500 further comprises pre-filtration zone 530 and filtration zone 550.

FIG. 6 depicts the structure of a rotary valve according to the embodiment of the cartridge depicted in FIG. 5. As can be seen, the exemplary rotary valve comprises eight ports. In use, various ports can align with conduits of the cartridge to bridge two or more conduits and create a fluid-tight connection between the conduits. As depicted in FIG. 6, ports 1 and 2 are permanently connected, as are ports 6 and 7. In practice, by rotating the rotary valve to the correct position, a fluid connection can be made between conduits connecting to ports 6 and 7, between conduits connecting to ports 1 and 2, or both. In this way, selection of the rotation position of a rotary valve can allow for fluid movement from selected conduits to other conduits, thus allowing for controlled movement of fluids through the cartridge. It is to be noted that the conduits are disposed within the cartridge in a manner that allows for movement of fluids in a controlled manner, and thus the rotary valve acts as a valve for opening and closing conduits, and in particular up to two conduits at one time. Each particular valve may be identical in design to one or more other valve on the cartridge, or may be different, bridging different ports or allowing for bridging of different numbers of ports. Each rotary valve may be designed in conjunction with the layout of conduits that it serves.

Turning now to FIGS. 5 and 6 in combination, operation of the cartridge for purification of RNA from white blood cells is described in detail. It is to be noted that reference will be made to elements depicted in FIG. 5, and that the four rotary valves depicted in FIG. 5 have structures either specifically as depicted in FIG. 6 or as can be derived from the structure depicted in FIG. 6. It is also to be understood that intake valves 511a-511j attach to various conduits that comprise fluids for use in the methods of the invention, whereas exit valve 511k attaches to a conduit for removal of waste materials from the cartridge. Removal of fluids may be accomplished by connecting exit port 511k to a source of vacuum to draw or pull out fluids in conjunction with any positive pushing pressure exerted on the fluids by way of intake ports 511a-511j. Furthermore, it is to be noted that FIG. 5 depicts rotary valves in certain particular positions, allowing for creation of bridges between particular conduits. One of skill in the art will immediately recognize that movement of the valve in a circular manner will allow for bridging of other conduits, as described in the text below. Thus, graphical depiction of each bridge discussed in the text need not be provided in the figures. It is also to be noted that all conduits may be pre-loaded or primed with appropriate fluid prior to use. In this way, accurate volumes of fluids can be measured and moved through the system.

As an example of use of the cartridge, purification of RNA from white blood cells in a whole blood sample, such as for example a 5 ml whole blood sample, is now discussed. A blood sample is inserted into cartridge 500 by way of sliding a blood sample container (e.g., a test tube) into sample receiving zone 520. Full insertion into sample receiving zone 520 causes puncture of the blood sample container by two needles (not shown). One needle is connected to conduit 571, which connects to air intake port 511g via a bridge connection through rotary valve 560. Air is caused to enter and pressurize the container, forcing blood from the container into conduit 572. Conduit 572 is connected to mixer 575 via a bridge completed by rotary valve 560. Just prior to entering and during traversal of mixer 575, blood mixes with Red Blood Cell (RBC) lysis solution pumped into cartridge 500 through conduit 573 from port 511c. Mixing begins the process of RBC lysis and aids in dispersion of the sample onto a pre-filter disposed at zone 530. The blood/RBC lysis mixture is caused to flow over and through the pre-filter, which results in entrapment of unlysed cells on the pre-filter. Upon complete loading of the blood onto the pre-filter, a series of volumes of RBC lysis buffer is exposed to the pre-filter, resulting in substantial to complete lysis of RBC entrapped on the filter and removal of RBC debris from the filter. During loading and lysing, waste material exits pre-filtration zone 530 through conduit 577 and ultimately exits cartridge 500 by way of a bridge created by valve 563 to conduit/vacuum trap 578. It is to be noted that any conduit desired, including but not limited to conduits 573, 577, and 584, can be “primed” or pre-filled with RBC lysis buffer prior to forcing blood from the container to the mixer, through conduit 577, and onto the pre-filter.

After sample has been loaded onto the pre-filter, and RBC substantially or completely lysed, the filter and entrapped material (predominantly WBC) are washed and dried through a series of washes with PBS followed by air purges. More specifically, PBS is caused to flow over and come in contact with the pre-filter/WBC complexes by way of intake port 511a and conduit 581 in a series of small-volume washes, each wash volume being purged from the pre-filtration zone by air introduced into the pre-filtration zone by way of intake port 511d or 511g and conduit 580, flowing through valve 560. In a preferred embodiment, eight cycles of washing/purging are performed, each with 1.5 ml of PBS or less. Waste fluid is removed as described above. Alternatively or in addition, the filter can be washed one or more times with water, introduced via inlet port 511b, conduit 582, valve 561, conduit 573′, and conduit 577.

Subsequent to washing and drying, WBC entrapped on the pre-filter are lysed by exposure to WBC lysis buffer introduced via intake port 511e and conduit 583. WBC lysis buffer can be exposed to WBC entrapped on the pre-filter as a continuous flow or in a series of two or more batches, allowing each batch of buffer to remain in contact with the pre-filter/cells for a pre-determined amount of time (e.g., 30 seconds) before removal and replacement by a subsequent batch. Lysis of cells causes large cell debris and DNA to become entrapped in the pre-filter, while allowing smaller molecules, including RNA to pass through. Cell lysate containing RNA as the predominant nucleic acid exits pre-filtration zone 530 via conduit 584 and proceed to static (Tesla) mixer 586 via a bridge created in valve 563. Concurrently, a composition comprising sulfolane is introduced into static mixer 586 via intake port 511f and conduit 585. The mixture is introduced into mixing chamber 592. Additional volumes of cell lysate may be introduced into static chamber 586 and mixing chamber 592 by exposing a continuous flow or a series (e.g., two or three) of batches of water to the pre-filter in the same fashion as described above for WBC lysis buffer. These water washes, which contain additional high-quality RNA, are mixed with sulfolane and introduced into mixing chamber 592 as described above. As fluids are introduced into mixing chamber 592, excess pressure is relieved by permitting air to escape via conduits 594, 595, and 596, via valves 561 and 562. The complete volume of lysate/wash can be introduced into mixing chamber 592 by an air purge over the pre-filtration zone according to fluid flow schemes discussed above. Water may be exposed to the pre-filter as described above.

Mixing in mixing chamber 592 is enhanced by introduction of air into the mixture by bubbling of the air through the mixture. In this regard, air is introduced into cartridge 500 via port 511g or 511d and conduit 580. It passes through valve 560, pre-filtration zone 530 and conduit 584 to valve 563, where it is shunted to conduit 584′ and into mixing chamber 592. As described above, air pressure is relieved via conduits 594, 595, and 596.

The mixture, which contains sulfolane and cell lysate comprising cellular RNA, is then exposed to a second filter unit, which is capable of binding the RNA in the mixture. To effect this binding, the mixture is forced from mixing chamber 592 by introduction of air from port 511g through valve 561 and conduit 594. The pressure caused by this air introduction forces the mixture to exit mixing chamber 592 through conduit 584′ and enter valve 563, where it is shunted to conduit 587, into filtration zone 550 and onto the filter (not depicted). Loading of the RNA onto the filter may be performed in a single continuous flow over the filter, or may be performed as a series of batches that are introduced and exposed for a period of time, then removed and replaced with a subsequent batch. Complete application of the mixture to the filter can be accomplished by allowing the pressurizing air to flow over the filter. Waste material exits filtration zone 550 via conduit 589 and valve 562, which creates a bridge between conduit 589 and conduit 596 or between conduit 589 and conduit 578.

The filter/RNA complex at filtration zone 550 is then washed with low salt buffer, either as a continuous stream or in a series of batches. Low salt buffer is introduced into cartridge 500 by way of intake port 511h and conduit 590 to valve 561. Valve 561 makes a bridge between conduit 590 and conduit 595, allowing low salt wash solution to travel to valve 563, where it is shunted to conduit 587 and onto the filter. Waste material exits filtration zone 550 as described above. Where a batch-type process is used, between washes, an air purge may be implemented. In this regard, air is introduced into cartridge 500 by way of port 511g and conduit 594a. At valve 561, conduit 594a is bridged to conduit 595, and air is supplied to filtration zone 550 as described for low salt wash solution, above.

After a final wash and optional air purge of the filter, which now comprises substantially pure RNA, the RNA is washed and dried a final time, with ethanol as a component of the final wash/dry composition. A composition comprising ethanol is introduced into cartridge 500 by way of port 511i and conduit 591. At valve 561, the ethanol is shunted to conduit 595 and proceeds to filtration zone 550 as described above. Waste is removed as above. At this juncture, the filter/RNA complex can be maintained in the ethanol for extended periods of time, and optionally shipped. For storage, valves are adjusted to close all pathways that allow ambient air to contact filtration zone 550, creating a sealed environment for the RNA. Further processing for various purposes may be performed on the stored RNA.

As an example, bound RNA may be eluted from the filter by exposure to water or a low ionic strength aqueous composition. In doing so, ethanol is removed by way of an air purge over filtration zone 550, as described above. A pre-determined amount of elution fluid, such as water, is then loaded or primed into the cartridge. To do so, water is introduced into cartridge 500 by way of port 511i and conduit 597. At valve 561, conduit 597 is bridged to conduit 595. Conduit 595 is bridged to conduit 578 at valve 563, which allows movement of water from intake port 511i to exit port 511k, and allows complete filling of conduits 597 and, particularly, 595. It is to be noted that the total volume of the pathway can be adjusted to a desired volume, such as 200 ul, to allow for automated elution of a known volume of RNA sample for each use of the cartridge. Where necessary, a chamber may be included as part of conduit 595 to adjust the volume to a desired value.

After pre-charging the lines with water, the water can be exposed to the filter/RNA complexes to cause RNA elution from the filter. To do so, air is introduced into cartridge 500 by way of intake port 511g and conduit 594a. At valve 561, conduit 594a is bridged to conduit 595, causing the water in conduit 595 to move through the conduit toward valve 563. At valve 563, conduit 595 is bridged to conduit 587 and the filter/RNA is contacted by the water. The water may be flowed over the filter in one continuous stream or may be left in contact with the filter for a pre-determined amount of time (e.g., 30 seconds, 1 minute). During loading of the water onto the filter, waste fluid, which is predominantly air but may include ethanol or other liquids, exits filtration zone 550 via valve 562 and conduit 578.

Eluted RNA is then collected in collection port 540 by causing the water/RNA composition to flow from filtration zone 550 to collection port 540 via conduit 589 and 598, a bridge between the two being made at valve 562. Conduit 598 terminates at a surface of collection port 540 that is above the liquid surface line of the water/RNA composition when the final volume of liquid is contained in the port. Typically, conduit 598 will terminate at a point that is in the top one-half of the height of collection port 540. By placing the entry point for the RNA at a point above the final liquid volume, RNA may be added to the collection port without significant bubbling or splattering of the composition. This improves reproducibility of the collection process and volumes achieved, while minimizing possible contamination of the exterior of the cartridge with sample. In essence, purified RNA is allowed to run down the side of collection port 540 to collect at the bottom.

It is to be understood that various modifications of the exemplary embodiments of the invention may be made to achieve a system according to the invention. For example, additional valves may be included in the cartridge of the system, for example to reduce or eliminate back-flow or other unwanted or unnecessary movement of fluids through channels of the cartridge. For example, a valve may be implemented that shuts off movement of blood from the receiving zone to conduit 572 after a pre-determined amount of time or after a pre-determined volume of blood has entered conduit 572. Likewise, rotary valves with a different number of valve ports or configuration of bridges between ports may be used to alter flow of fluids through the cartridge or to reduce or increase the number of conduits on the cartridge. Likewise, for example, solenoids can be used as part of the cartridge or as an additional component of the system to control movement of fluids into and out of intake and exit ports of the cartridge. Further, additional conduits may be introduced to eliminate the use of a single conduit for movement of multiple fluids. For example, one or more additional conduits may be implemented to allow isolation of air and/or water movement from movement of other fluids. Other variations will be apparent to those of skill in the art upon consideration of the above disclosure, the appended drawings, and the following claims. All such variations are to be considered as encompassed by the present invention.

1. An article of manufacture for purification of RNA from white blood cells of a sample comprising white blood cells, said article comprising:
at least one port for intake of each of a number of fluids;
at least one port for exit of at least one fluid;
at least one solid support for binding of cells of whole blood;
at least one solid support for binding of RNA from cells of whole blood; and
at least one rotary valve for control of movement of fluids among three or more conduits,
wherein the intake port(s), exit port(s), solid supports, and rotary valve(s) are connected to each other to create a circuit from the intake port to the exit port.

2. The article of claim 1, wherein the sample comprises whole blood of an animal.

3. The article of claim 2, wherein the animal is a human.

4. The article of claim 1, which comprises ten intake ports and one exit port.

5. The article of claim 1, which comprises four rotary valves that function independently of each other.

6. The article of claim 1, wherein the rotary valve(s) are computer controllable.

7. The article of claim 1, wherein the solid support for binding of RNA is a glass fiber filter.

8. An automated method for the isolation of RNA from white blood cells, said method comprising:
a) causing a sample comprising white blood cells to contact and flow over a first solid support, whereby the first solid support entraps cells present in the sample and removes them from the sample;
b) causing cells other than white blood cells on the first solid support to lyse, whereby lysis causes the cells and their components to be released from the first solid support and removed from cells remaining entrapped by the first solid support;
c) causing the cells remaining on the first solid support to lyse, thereby releasing RNA into a lysate; and
d) causing the lysate to contact and flow over a second solid support, whereby the second solid support binds the RNA and allows other substances to pass unbound,
wherein the method is performed on a single device and the movement of fluids within the method is controlled, at least in part, by two or more rotary valves present on the device, and
wherein all of the steps of the method are controlled automatically by a computing means.

9. The method of claim 8, further comprising:
causing the bound RNA to elute from the second solid substrate; and
collecting the eluted RNA.

10. The method of claim 9, wherein the method comprises:
between the steps of binding of RNA to the second solid substrate and causing the bound RNA to elute from the second solid substrate, exposing the bound RNA to a liquid comprising ethanol,
wherein the method does not include exposing the bound RNA to an aqueous composition between exposing it to an ethanol-containing liquid and eluting it from the second solid substrate.

11. The method of claim 10, wherein RNA bound to the second solid substrate is treated with ethanol then eluted with water or a low ionic strength aqueous solution, without an intervening wash with an aqueous composition.

12. The method of claim 8, wherein the sample comprises whole blood.

13. The method of claim 8, wherein the white blood cells are cultured or transformed white blood cells.

14. The method of claim 8, wherein the method is performed in 15 minutes or less.

15. The method of claim 8, wherein movement of fluids is automatically controlled by the computing means at least in part by regulating positive pressure to push the fluids and negative pressure to pull fluids.

16. The method of claim 8, wherein movement of fluids is automatically controlled by the computing means at least in part by regulating the positioning of the rotary valves to allow selective opening and closing of conduits for movement of fluids through the device.

17. The method of claim 8, wherein the device comprises four rotary valves.

18. The method of claim 8, wherein the method comprises:
mixing whole blood with a composition that causes lysis of red blood cells;
exposing the mixture to a first solid support that entraps blood cells;
exposing the entrapped blood cells to a composition that causes lysis of red blood cells to effect lysis of a substantial amount of red blood cells entrapped on the first solid support;
washing the first solid support at least one time with an aqueous solution to remove entrapped cell debris and blood components other than white blood cells;
optionally, passing a gas over the first solid substrate to effect partial or total drying of the substrate;
exposing the entrapped white blood cells to a composition that causes the white blood cells to lyse;
collecting the lysate;
exposing the first solid substrate to water to elute additional cell lysis substances;
collecting the water-containing composition;
mixing the lysate and water-containing composition;
combining the lysate-water mixture with sulfolane to make an RNA binding solution;
exposing the RNA binding solution to a second solid substrate, which binds the RNA in the solution;
optionally exposing the bound RNA to an aqueous solution to wash off impurities;
exposing the bound RNA to ethanol.

19. The method of claim 18, further comprising storing the bound RNA in ethanol for at least one day.

20. The method of claim 18, further comprising exposing the bound RNA to water or an aqueous solution to elute the RNA from the second solid substrate.

21. A system for purification of RNA from white blood cells of a sample comprising white blood cells, said system comprising:
the article of manufacture of claim 1;
at least one pump for movement of fluids into, through, and out of the article of manufacture;
a reagent pack for storing fluids to be moved through the article of manufacture; and
a computer for controlling movement of fluids through the article of manufacture.

22. The system of claim 21, wherein the pump comprises more than one head and is capable of creating both a positive pressure on a fluid and a negative pressure on a fluid.

23. The system of claim 21, wherein the pump is a peristaltic pump.

24. The system of claim 21, wherein the reagent pack comprises two or more containers for containing two or more different fluids, each of which containers is capable of being connected to a port in the article of manufacture by way of a connector.

25. The system of claim 21, wherein the computer controls the movement of the pump(s) and controls movement of valves on the article of manufacture.

26. The system of claim 21, further comprising a waste pack for receiving and containing waste from the article of manufacture.
